(19) United States
(12) Patent Application Publication
(10) Pub. No.: US 2025/0341599 A1
(43) Pub. Date: Nov. 6, 2025

(54) AN EFFICIENT APPROACH TO OPTIMAL EXPERIMENTAL DESIGN FOR MAGNETIC RESONANCE FINGERPRINTING WITH B-SPLINES

(71) Applicant: Board of Regents, The University of Texas System, Austin, TX (US)

(72) Inventors: Bo Zhao, Austin, TX (US); Evan Scope Crafts, Austin, TX (US)

(21) Appl. No.: 18/860,838

(22) PCT Filed: Apr. 28, 2023
(86) PCT No.: PCT/US2023/066381

§ 371 Date: Oct. 28, 2024

Related U.S. Application Data
(60) Provisional application No. 63/336,538, filed on Apr. 29, 2022.

(51) Int. Cl. G01R 33/54; A61B 5/055; G01R 33/48

(52) U.S. Cl. CPC G01R 33/543; A61B 5/055; G01R 33/4828

(57) ABSTRACT

A method of performing a diagnostic scan of a subject comprises defining a set of data acquisition parameter sequences, defining a set of upper bounds and a set of lower bounds for each of the set of data acquisition parameter sequences, defining a set of representative tissue parameters for a tissue of the subject, selecting a set of basis functions, calculating values for a set of basis function coefficients to yield a piecewise polynomial representation of each of the set of data acquisition parameter sequences within the sets of upper and lower bounds based on the set of desired tissue parameters, and performing a diagnostic scan of the subject using the calculated data acquisition parameter sequences.

### CROSS-REFERENCE TO RELATED APPLICATIONS

This application claims priority to U.S. Provisional Patent Application No. 63/336,538 filed on Apr. 29, 2022, incorporated herein by reference in its entirety.

### STATEMENT REGARDING FEDERALLY SPONSORED RESEARCH OR DEVELOPMENT

This invention was made with government support under Grant no. R00 EB027181 awarded by the National Institutes of Health. The government has certain rights in the invention.

### BACKGROUND OF THE INVENTION

MR Fingerprinting is an emerging quantitative MRI technique that enables simultaneous acquisition of multiple MR tissue parameter maps (e.g., T₁, T₂, and spin density) in a single imaging experiment (Ma et al., Magnetic resonance fingerprinting. Nature 2013; 495:187-192.). It holds the great potential of transforming quantitative MRI, and has found a number of promising applications (e.g., (Chen et al., MR fingerprinting for rapid quantitative abdominal imaging. Radiology 2016; 279:278-286; Badve et al., MR fingerprinting of adult brain tumors: Initial experience. Am J Neuroradiol 2017; 38:492-499; Yu et al., Development of a combined MR fingerprinting and diffusion examination for prostate cancer. Radiology 2017; 283:729-738; Liu Y et al., Cardiac magnetic resonance fingerprinting: Technical overview and initial results. JACC: Cardiovasc Imaging 2018; 11:1837-1853; Chen et al., Three-dimensional MR fingerprinting for quantitative breast imaging. Radiology 2019; 290:33-40; Cloos et al., Rapid radial T1 and T2 mapping of the hip articular cartilage with magnetic resonance fingerprinting. J Magn Reson Imaging 2019; 50:810-815; Kiselev et al., Toward quantification: Microstructure and magnetic resonance fingerprinting. Investig Radiol 2021; 56:1-9; Jaubert et al., T1, T2, and fat fraction cardiac MR fingerprinting: Preliminary clinical evaluation. J Magn Reson Imaging 2021; 53:1253-1265)). The original MR Fingerprinting acquisition features the use of a transient-state encoding scheme with random or pseudorandom acquisition parameters to probe the spin system (Ma et al., Magnetic resonance fingerprinting. Nature 2013; 495:187-192). This generates unique magnetization evolutions for different tissues of interest, which are used for a subsequent pattern-matching based parameter estimation.

More information about MR Fingerprinting and related methods may be found in U.S. Pat. No. 10,241,173, issued on Mar. 26, 2019, and U.S. Pat. No. 10,241,176, issued on Mar. 26, 2019, both of which are incorporated herein by reference in their entirety.

The use of randomized parameter encoding has led to promising initial results. However, it has been shown that this scheme has sub-optimal signal-to-noise ratio (SNR) efficiency from a statistical inference perspective (Zhao et al., Optimal experiment design for magnetic resonance fingerprinting. In: Proc. IEEE Eng. Med. Bio. Conf., 2016. pp. 453-456; Zhao et al., Towards optimized experiment design for magnetic resonance fingerprinting. In: Proc. Int. Symp. Magn. Reson. Med., 2016. p. 2835; Zhao et al., Optimal experiment design for magnetic resonance fingerprinting: Cramér-Rao bound meets spin dynamics. IEEE Trans Med Imaging 2019; 38:844-861). The optimization of acquisition parameters for MR Fingerprinting is naturally framed as an optimal experimental design (OED) problem, which has been previously employed in optimizing steady-state quantitative MRI sequences (e.g., (Jones et al., Optimal sampling strategies for the measurement of spin-spin relaxation times. J Magn Reson 1996; 113:25-34; Deoni et al., Determination of optimal angles for variable nutation proton magnetic spin-lattice, T1, and spin-spin, T2, relaxation times measurement. Magn Reson Med 2004; 51:194-199; Fleysher et al., Optimizing the precision-per-unit-time of quantitative MR metrics: examples for T1, T2, and DTI. Magn Reson Med 2007; 57:380-387; Akçakaya et al., On the selection of sampling points for myocardial T1 mapping. Magn Reson Med 2015; 73:1741-1753; Whitaker et al., Myelin water fraction estimation using small-tip fast recovery MRI. Magn Reson Med 2020; 84:1977-1990)). In the context of transient-state imaging, an OED framework utilizing an estimation-theoretic bound, i.e., the Cramer-Rao bound (CRB), was introduced in (Zhao et al., Optimal experiment design for magnetic resonance fingerprinting. In: Proc. IEEE Eng. Med. Bio. Conf., 2016. pp. 453-456; Zhao et al., Towards optimized experiment design for magnetic resonance fingerprinting. In: Proc. Int. Symp. Magn. Reson. Med., 2016. p. 2835; Zhao et al., IEEE Trans Med Imaging 2019; 38:844-861) to optimize acquisition parameters of MR Fingerprinting. This framework enables better estimation performance, and, in particular, it significantly improves the estimation accuracy for the T₂ parameter, which helps address an inherent limitation of the MR Fingerprinting technique. In addition, it was observed that the optimized acquisition sequences from this framework are highly structured rather than random or pseudo-random as in (Ma et al., Magnetic resonance fingerprinting. Nature 2013; 495:187-192).

Following the early work, a number of extensions have been developed in the OED framework (Maidens et al., Parallel dynamic programming for optimal experiment design in nonlinear systems. In: Proc. IEEE Conf. Decis. Control, 2016. pp. 2894-2899; Lee et al., Buonincontri G, Hargreaves B A, Flexible and efficient optimization of quantitative sequences using automatic differentiation of Bloch simulations. Magn Reson Med 2019; 82:1438-1451; Assländer et al., Cloos M A, Optimized quantification of spin relaxation times in the hybrid state. Magn Reson Med 2019; 82:1385-1397; Lahiri et al., Optimizing MRF-ASL scan design for precise quantification of brain hemodynamics using neural network regression. Magn Reson Med 2020; 83:1979-1991; Heesterbeek et al., Sequence optimisation for multi-component analysis in magnetic resonance fingerprinting. In: Proc. Int. Symp. Magn. Reson. Med., 2021. p. 1561). For example, Maiden et al developed a dynamic programming algorithm to solve the optimal experimental design (OED) problem (Maidens et al., Parallel dynamic programming for optimal experiment design in nonlinear systems. In: Proc. IEEE Conf. Decis. Control, 2016. pp. 2894-2899). Lee et al applied automatic differentiation to simplify the gradient calculation for the experimental design (Lee et al., Buonincontri G, Hargreaves B A, Flexible and efficient optimization of quantitative sequences using automatic differentiation of Bloch simulations. Magn Reson Med 2019; 82:1438-1451). Assländer et al applied the OED framework to optimize flip-angle sequences for magnetization dynamics in the hybrid state (Assländer et al., Cloos M A, Optimized quantification of spin relaxation times in the hybrid state. Magn Reson Med 2019; 82:1385-1397). Lahiri et al incorporated a neural network to solve the OED problem for an advanced neuroimaging application (Lahiri et al., Optimizing MRF-ASL scan design for precise quantification of brain hemodynamics using neural network regression. Magn Reson Med 2020; 83:1979-1991). Heesterbeek et al applied an OED-based approach to MR Fingerprinting with a multi-component tissue biophysical model (Heesterbeek et al., Sequence optimisation for multi-component analysis in magnetic resonance fingerprinting. In: Proc. Int. Symp. Magn. Reson. Med., 2021. p. 1561).

Despite the improved estimation performance, the OED problem for MR Fingerprinting is often computationally expensive to solve, which impairs its practical utility. Disclosed herein is an efficient approach to solving the above OED problem, which is motivated by the early observation that the optimized encoding scheme for MR Fingerprinting is highly structured rather than random or pseudorandom when using the formulation in (Zhao et al., IEEE Trans Med Imaging 2019; 38:844-861). More specifically, the optimized acquisition parameter sequences are in some embodiments approximately piecewise polynomials and, in particular, the optimized repetition time sequences exhibit piecewise binary structure.

Thus, there is a need in the art for a computationally inexpensive approach to solving the OED problem for MR Fingerprinting that optimizes SNR while enabling better estimation performance and estimation accuracy.

### SUMMARY OF THE INVENTION

In one aspect, a method of performing a diagnostic scan of a subject comprises defining a set of data acquisition parameter sequences, defining a set of upper bounds and a set of lower bounds for each of the set of data acquisition parameter sequences, defining a set of representative tissue parameters for a tissue of the subject, selecting a set of basis functions, calculating values for a set of basis function coefficients to yield a piecewise polynomial representation of each of the set of data acquisition parameter sequences within the sets of upper and lower bounds based on the set of desired tissue parameters, and performing a diagnostic scan of the subject using the calculated data acquisition parameter sequences.

In one embodiment, the diagnostic scan is a magnetic resonance imaging (MRI) scan. In one embodiment, the diagnostic scan is a magnetic resonance fingerprinting scan. In one embodiment, the set of data acquisition parameter sequences are selected from flip angles, radiofrequency phases, repetition times, echo times, time-bandwidth products, or k-space sampling locations. In one embodiment, the set of basis functions is selected from B-splines, wavelets, radial basis functions, or Fourier basis functions. In one embodiment, the set of basis functions is a B-spline basis function.

In one embodiment, the method further comprises the step of selecting a degree of the B-spline basis functions to use in the step of calculating the piecewise polynomial. In one embodiment, the degree of the B-spline basis function is selected using a machine learning algorithm. In one embodiment, the method further comprises the steps of generating a set of possible values for each of the set of basis function coefficients, iterating through each combination of the possible values in the set of possible values to generate a set of piecewise polynomials, calculating a result of a simulated diagnostic scan for each piecewise polynomial in the set of piecewise polynomials to create a multidimensional set of results, and calculating a maximum value among the multidimensional set of results using an algorithm to yield the piecewise polynomial representation, wherein the step of calculating the values of the set of basis function coefficients to yield the piecewise polynomial comprises executing an iterative algorithm.

In one embodiment, the iterative algorithm is selected from a sequential quadratic programming algorithm, an interior point algorithm, a genetic algorithm, and a simulated annealing algorithm. In one embodiment, the algorithm is a sequential quadratic programming algorithm. In one embodiment, the algorithm comprises setting a tolerance threshold and terminating the algorithm when the norm of a gradient of the multidimensional set of results is less than the tolerance threshold. In one embodiment, the simulated diagnostic scan is a Bloch simulation. In one embodiment, the method further comprises the step of selecting a set of time offsets for the set of basis functions to calculate the piecewise polynomial. In one embodiment, the tissue of the subject is selected from liver, spleen, kidney medulla, kidney cortex, kidney, skeletal muscle, fat, myocardium, abdomen, lungs, stomach, intestines, brain, or blood. In one embodiment, the step of defining the set of upper and lower bounds comprises calculating at least one of the upper and lower bound based on a desired total acquisition time or a specific absorption rate. In one embodiment, the step of calculating values for each of the set of basis function coefficients to yield a piecewise polynomial representation of each of the set of data acquisition parameters comprises constructing the piecewise polynomials using the Cox-de Boor recursion formula.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a diagram of an exemplary computing device.

FIG. 2A, FIG. 2B and FIG. 2C are diagrams of B-spline basis functions.

FIG. 3A, FIG. 3B, FIG. 3C and FIG. 3D are diagrams of the flip angle sequence and repetition time sequence.

FIG. 4A, FIG. 4B, FIG. 4C and FIG. 4D are diagrams of magnetization dynamics for the representative white matter and gray matter tissues.

FIG. 5A, FIG. 5B, FIG. 5C, and FIG. 5D are diagrams of the flip angle sequence and repetition time sequence.

FIG. 6A and FIG. 6B depict the reconstructed T₁ and T₂ maps and the corresponding error maps of the disclosed approach.

FIG. 7A and FIG. 7B depict the reconstructed T₁ and T₂ maps and the corresponding error maps of the disclosed approach.

FIG. 8A and FIG. 8B depict the bias and variance of the reconstructed T₁ and T₂ maps from the MC simulations.

FIG. 9A and FIG. 9B are diagrams of reconstructed T₁ parameter maps and normalized standard deviation maps for the phantom experiments.

FIG. 10A and FIG. 10B are diagrams of reconstructed T₂ parameter maps and normalized standard deviation maps for the phantom experiments.

FIG. 11A and FIG. 11B are diagrams of reconstructed T₁ parameter maps and normalized standard deviation maps for the in vivo experiments.

FIG. 12A and FIG. 12B are diagrams of reconstructed T₂ parameter maps and normalized standard deviation maps for the in vivo experiments.

FIG. 13A, FIG. 13B, and FIG. 13C show the resulting optimized FA and TR sequences and associated representative magnetization dynamics from the disclosed approach at N=400.

FIG. 14A and FIG. 14B show the corresponding reconstructed T₁ and T₂ maps from numerical simulations with N=400 and SNR=33 dB.

FIG. 15A, FIG. 15B, and FIG. 15C are diagrams of acquisition parameters and associated magnetization dynamics.

FIG. 16A and FIG. 16B show reconstructed T₁ and T₂ maps.

FIG. 17 shows the optimized FA and TR sequences using the above initializations as well as the cost function values associated with these solutions.

FIG. 18A and FIG. 18B show the reconstructed T₁ and T₂ maps using the acquisition sequences in FIG. 17 at acquisition length N=300 and SNR=33 dB.

FIG. 19 shows the resulting overall relative reconstruction errors for T₁ and T₂. using the OED with B-spline basis function subspaces of dimension L_{α}=L_{TR}=20,40, and 200 and acceleration factors AF=1, 16, 24, and 48 at N=400 and SNR=33 dB.

FIG. 20 shows the optimized acquisition parameters from the SQP algorithm, the interior-point algorithm, and the genetic algorithm.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

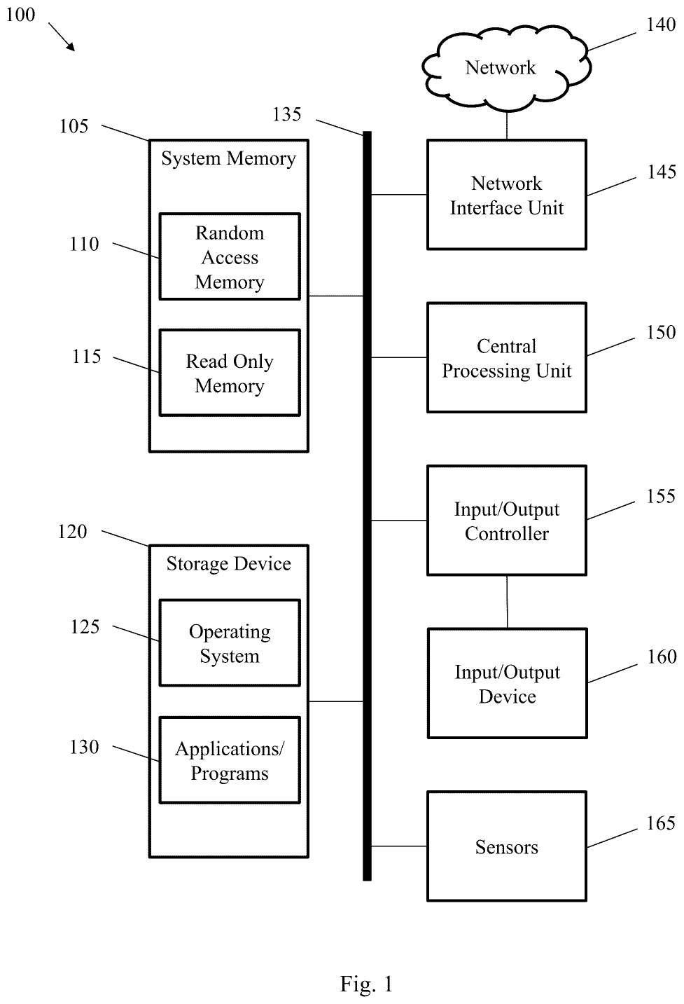

FIG. 1 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 1 depicts an illustrative computer architecture for a computer 100 for practicing the various embodiments of the invention. The computer architecture shown in FIG. 1 illustrates a conventional personal computer, including a central processing unit 150 (“CPU”), a system memory 105, including a random access memory 110 (“RAM”) and a read-only memory (“ROM”) 115, and a system bus 135 that couples the system memory 105 to the CPU 150. A basic input/output system containing the basic routines that help to transfer information between elements within the computer, such as during startup, is stored in the ROM 115. The computer 100 further includes a storage device 120 for storing an operating system 125, application/program 130, and data.

The storage device 120 is connected to the CPU 150 through a storage controller (not shown) connected to the bus 135. The storage device 120 and its associated computer-readable media provide non-volatile storage for the computer 100. Although the description of computer-readable media contained herein refers to a storage device, such as a hard disk or CD-ROM drive, it should be appreciated by those skilled in the art that computer-readable media can be any available media that can be accessed by the computer 100.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 100 may operate in a networked environment using logical connections to remote computers through a network 140, such as TCP/IP network such as the Internet or an intranet. The computer 100 may connect to the network 140 through a network interface unit 145 connected to the bus 135. It should be appreciated that the network interface unit 145 may also be utilized to connect to other types of networks and remote computer systems.

The computer 100 may also include an input/output controller 155 for receiving and processing input from a number of input/output devices 160, including a keyboard, a mouse, a touchscreen, a camera, a microphone, a controller, a joystick, or other type of input device. Similarly, the input/output controller 155 may provide output to a display screen, a printer, a speaker, or other type of output device. The computer 100 can connect to the input/output device 160 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 120 and/or RAM 110 of the computer 100, including an operating system 125 suitable for controlling the operation of a networked computer. The storage device 120 and RAM 110 may also store one or more applications/programs 130. In particular, the storage device 120 and RAM 110 may store an application/program 130 for providing a variety of functionalities to a user. For instance, the application/program 130 may comprise many types of programs such as a word processing application, a spreadsheet application, a desktop publishing application, a database application, a gaming application, internet browsing application, electronic mail application, messaging application, and the like. According to an embodiment of the present invention, the application/program 130 comprises a multiple functionality software application for providing word processing functionality, slide presentation functionality, spreadsheet functionality, database functionality and the like.

The computer 100 in some embodiments can include a variety of sensors 165 for monitoring the environment surrounding and the environment internal to the computer 100. These sensors 165 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

In some embodiments, the disclosed systems and methods introduce an efficient approach to the OED problem for MR Fingerprinting. Specifically, the disclosed approach represents the acquisition parameter sequences with a special class of piecewise polynomial functions, i.e., B-spline basis functions, which constrains the acquisition parameters to low-dimensional subspaces. This reduces the search space of the OED problem, thereby improving computational efficiency. Moreover, due to the rich representations of B-spline basis functions, the disclosed approach allows for the incorporation of prior knowledge regarding the structure of different acquisition parameters, which facilitates the experimental design.

Some embodiments of the disclosed systems and methods significantly improve the computational efficiency over the state-of-the-art approaches in (Zhao et al., IEEE Trans Med Imaging 2019; 38:844-861), while offering a comparable SNR efficiency benefit. For MR Fingerprinting experiments with a typical acquisition length, the OED problem can be solved in approximately one minute or less. Representative results from numerical simulations, phantom experiments, and in vivo experiments are shown to demonstrate the performance of the disclosed approach. The preliminary results of this work were presented in (Scope & Zhao, An efficient approach to optimal design of MR fingerprinting experiments with B-spline basis functions. In: Proc. Int. Symp. Magn. Reson. Med., 2021. p. 1566). The disclosed system is the first work that utilizes splines to solve the OED problem with the Cramér-Rao bound (CRB).

The OED framework determines the data acquisition parameters of MR Fingerprinting experiments by maximizing some utility function, such as the SNR efficiency or the information content of an imaging experiment, subject to a set of design constraints. Mathematically, the OED problem for MR Fingerprinting can be formulated as

$${max}_{U} Ψ ( U ) \qquad Equation 1$$

$$s . t . U ∈ 𝒰 ,$$

where U∈ [symbol] ^{N×D} is the design matrix that encompasses the data acquisition parameters that are optimized in an MR Fingerprinting experiment, ψ(·): [symbol] ^{N×D}→ [symbol] is the design metric that measures the utility of a specific experimental design, [symbol] ⊂ [symbol] ^{N×D} is the set that contains all feasible data acquisition parameters, N denotes the length of the imaging experiment, and D denotes the number of design acquisition parameters for each repetition time.

In the above OED formulation, there are three key ingredients: (a) the design matrix U; (b) the design metric ψ(·); and (c) the constraint set [symbol] . Next, these components are described for the disclosed approach.

The design matrix U∈ [symbol] ^{N×D} contains the data acquisition parameters that are optimized for an MR Fingerprinting experiment. Each column of U is associated with one acquisition parameter sequence, e.g., flip angle (FA), radiofrequency (RF) phase, repetition time (TR), and echo time (TE) sequences, as well as parameters characterizing the RF pulse envelope, for example the time-bandwidth product, and/or parameters characterizing the spatial encoding, for example the k-space sampling locations. For simplicity in the foregoing examples and discussion, it is assumed that the flip angles and repetition times are the design acquisition parameters. The generalization to other design parameters is mathematically straightforward. Accordingly, U=[U_{α}, U_{TR}]∈ [symbol] ^{N×2}, where U_{α}=[α₁, . . . , α_{N}]^{T} ∈ [symbol] ^{N} and U_{TR}=[TR₁, . . . , TR_{N}]^{T} ∈ [symbol] ^{N} denote the FA sequence and the TR sequence, respectively.

The design metric Ψ(·) is dictated by the goal of the experimental design. The present disclosure adopts the CRB-based A-optimality criterion, as in (Zhao et al., Optimal experiment design for magnetic resonance fingerprinting. In: Proc. IEEE Eng. Med. Bio. Conf., 2016. pp. 453-456; Zhao et al., Towards optimized experiment design for magnetic resonance fingerprinting. In: Proc. Int. Symp. Magn. Reson. Med., 2016. p. 2835; Zhao et al., IEEE Trans Med Imaging 2019; 38:844-861), which essentially measures the SNR efficiency of an imaging experiment. Mathematically, the design metric can be written as follows:

$$Ψ ( U ) = - {\sum }_{l = 1}^{L} tr ( {W}^{( l )} {C}^{1 / 2} ( {θ}^{( l )} , U ) ) . \qquad Equation 2$$

Here {θ^{(l)}}ᵢ₌₁^{L} is a set of representative tissues that are assumed to be known for a specific application of interest (e.g., neuroimaging), where θ∈ [symbol] ^{P×1} (e.g., θ^{(l)}=[T₁^{(l)}, T₂^{(l)}, M₀^{(l)}]^{T}∈ [symbol] ³); C(θ, U)∈ [symbol] ^{P×P} denotes the CRB matrix; tr(·) computes the trace of a matrix; and each W^{(l)} is a diagonal weighting matrix with W_{p,p}^{(l)}=ωₚ/θₚ^{(l)}≥0 incorporating application-specific design considerations through the choice of the non-negative parameters {ωₚ}ₚ₌₁^{P}. As would be understood by one skilled in the art, the values of the weighting matrix or matrices may be adjusted based on the goal of the imaging experiment or diagnostic imaging. For example, if the primary goal of the imaging experiment or diagnostic imaging was to acquire Tₗ tissue parameter maps, then that component could be given a higher weight.

Although various embodiments described herein may be presented in the context of systems and methods for imaging one or more specific tissues or body regions, it is understood that the systems and methods disclosed herein may be used to image any suitable tissue, body region, partial tissue, partial body region, or combination thereof. These include, but are not limited to, the liver, spleen, kidney medulla, kidney cortex, kidney, skeletal muscle, fat, myocardium, abdomen, lungs, stomach, intestines, brain, and/or blood.

The constraint set [symbol] incorporates physical considerations and considerations from parameter decoding into the experimental design. For example, the early work (Zhao et al., IEEE Trans Med Imaging 2019; 38:844-861) imposes the following constraint in the OED problem:

$$\tilde{𝒰} = \{ {U}_{α} , {U}_{TR} ∈ {ℝ}^{N} \vert \begin{matrix}{α}^{min} ≼ {U}_{α} ≼ {α}^{max} , & T {R}^{min} ≼ {U}_{TR} ≼ T {R}^{max} , \\ \text{\textbar Dα\textbar} ≼ Δ {α}^{max} . & \end{matrix} \} . \qquad Equation 3$$

Here αₘₐₓ, αₘᵢₙ ∈ [symbol] ^{N} respectively contain the upper bounds and lower bounds on the individual FAs, TR^{max}, TR^{min} ∈ [symbol] ^{N} respectively contain the upper bounds and lower bounds on the individual TRs, Δα^{max} ∈ [symbol] ^{N-1} contains the tolerance on the maximum flip angle variation, and D∈ [symbol] ^{N-1×N} denotes the first-order finite difference matrix such that [D_{α}]ᵢ=αᵢ₊₁−αᵢ. In Equation 3, [symbol] denotes componentwise inequality, and |·| takes the componentwise absolute value of a vector.

In Equation 3, the upper and lower bounds on the FAs and TRs address the physical considerations of the experimental design, such as total acquisition time and specific absorption rate (SAR), while the constraint on the flip angle variation essentially controls magnetization evolutions, which is advantageous to parameter estimation from highly-undersampled k-space data. It has been demonstrated in (Zhao et al., IEEE Trans Med Imaging 2019; 38:844-861) that the experiments from the OED problem with [symbol] enable a substantial improvement of the estimation accuracy over conventional MR Fingerprinting experiments (Ma et al., Magnetic resonance fingerprinting. Nature 2013; 495:187-192; Jiang et al., MR fingerprinting using fast imaging with steady state precession (FISP) with spiral readout. Magn Reson Med 2015; 74:1621-1631). Furthermore, it was observed that highly structured acquisition parameters demonstrated significantly improved results over random or pseudorandom acquisition parameters as in conventional MR Fingerprinting experiments (Ma et al., Magnetic resonance fingerprinting. Nature 2013; 495:187-192; Jiang et al., Magn Reson Med 2015; 74:1621-1631). More specifically, the structured acquisition parameters are approximately piecewise polynomials, and, in particular, the TR sequences calculated using the disclosed methods exhibit a binary structure.

In general, the choice of bounds is dependent on considerations from a particular imaging experiment that are not incorporated into the problem objective function. For example, in one embodiment the bounds on a flip angle magnitude may account for physical considerations during the imaging experiment, such as constraints on the specific absorption rate (SAR) and may for example be set to [10 degrees, 60 degrees]. In one example, the constraints on the repetition time limit and the total duration of the experiment and were set to [11 ms, 15 ms]. For different imaging applications, different SAR considerations could lead to different choices of bounds on the flip angle, while considerations regarding total experiment time could lead to different choices of repetition time bounds.

Despite the improved estimation performance, the above OED problem is often computationally expensive to solve, which limits its practical utility. The present disclosure provides an alternative constraint set that significantly improves computational efficiency without compromising estimation accuracy. Specifically, leveraging the special structure of the optimized acquisition sequences, a set of novel subspace constraints are incorporated into the OED problem, which significantly reduces its search space. The disclosed constraints assume that the acquisition parameters of interest lie in function spaces that consist of piecewise polynomials. B-spline basis functions are used in some embodiments disclosed herein to represent these acquisition parameters. B-spline basis functions are sets of basis functions for the above function spaces (Höllig & Hörner, Approximation and Modeling with B-Spline basis functions. Philadelphia, PA: Society for Industrial and Applied Mathematics, 2013). B-spline basis functions have many desirable properties (e.g., local support, controlled smoothness, and translation-invariance), and have been widely used in many fields, including approximation theory (Schumaker L, Spline Functions: Basic Theory. Cambridge: Cambridge University Press, 2007), scientific computing (Heath M T, Scientific Computing: An Introductory Survey. Philadelphia, PA: Society for Industrial and Applied Mathematics, 2018), machine learning (Hastie et al., The Elements of Statistical Learning: Data Mining, Inference, and Prediction. New York: Springer, 2009; Fey et al., SplineCNN: Fast geometric deep learning with continuous B-spline kernels. In: Proc. IEEE/CVF Conf. Comput. Vis. Pattern Recognit., 2018. pp. 869-877), signal and image processing (Unser et al., B-spline signal processing: Part I-theory. IEEE Trans Signal Process 1993; 41:821-833; Unser et al., B-spline signal processing: Part II-efficient design and applications. IEEE Trans Signal Process 1993; 41:834-848; Unser M, Splines: A perfect fit for signal and image processing. IEEE Signal Process Mag 1999; 16:22-38), and medical imaging (Rueckert et al., Nonrigid registration using free-form deformations: Application to breast MR images. IEEE Trans Med Imaging 1999; 18:712-721; Hao et al., Joint design of excitation k-space trajectory and RF pulse for small-tip 3D tailored excitation in MRI. IEEE Trans Med Imaging 2016; 35:468-479).

Specifically, in some embodiments disclosed herein, the acquisition parameter sequences, i.e., {αₙ}=ₙ₌₁^{N} and {TRₙ}ₙ₌₁^{N}, are treated as samples from two piecewise polynomials α(t) and TR(t) on the uniform grid [1, 2, . . . , N], while assuming that α(t) and TR(t) can be represented using the B-spline basis functions as follows:

$$α ( t ) = {\sum }_{l = 1}^{{L}_{α}} {g}_{l} {B}_{l}^{{d}_{α}} ( t ) , \qquad Equation 4$$

$$T R ( t ) = {\sum }_{l = 1}^{{L}_{TR}} {h}_{l} {B}_{l}^{{d}_{TR}} ( t ) \qquad Equation 5$$

Here {Bₗ^{d}^{_{α}}(t)} and {Bₗ^{d}^{_{TR}}(t)} are the B-spline basis functions for α(t) and TR(t), respectively, and {gₗ}ₗ₌₁^{L}^{_{α}} and {hₗ}ₗ₌₁^{L}^{_{TR}} are the corresponding B-spline coefficients. The two sets of B-spline basis functions are determined by the degrees of the piecewise polynomials they represent, denoted as d_{α} and d_{TR}, and sequences of increasing scalars, i.e., t_{α} and t_{TR}, known as knots that determine the connecting points of the piecewise polynomials. Given the degree d and the knot sequence t₀<t₁< . . . <tₘ, the piecewise polynomials can be efficiently constructed through the Cox-de Boor recursion formula (Scope & Zhao, An efficient approach to optimal design of MR fingerprinting experiments with B-spline basis functions. In: Proc. Int. Symp. Magn. Reson. Med., 2021. p. 1566; de Boor C, On calculating with B-splines. J Approx Theory 1972; 6:50-62):

$${B}_{l}^{d} ( t ) = \frac{t - {t}_{l}}{{t}_{l + d} - {t}_{l}} {B}_{l}^{d - 1} ( t ) + \frac{{t}_{l + d + 1} - t}{{t}_{l + d + 1} - {t}_{l + 1}} {B}_{l + 1}^{d - 1} ( t ) , for l = 1 , ... , L , \qquad Equation 6$$

with the initialization Bₗ⁰(t) (i.e., B-spline basis functions of degree 0) defined as:

$${B}_{l}^{0} ( t ) = \{ \begin{matrix}1 & if {t}_{l} \leq t \leq {t}_{t + 1} , \\ 0 & otherwise .\end{matrix} \qquad Equation 7$$

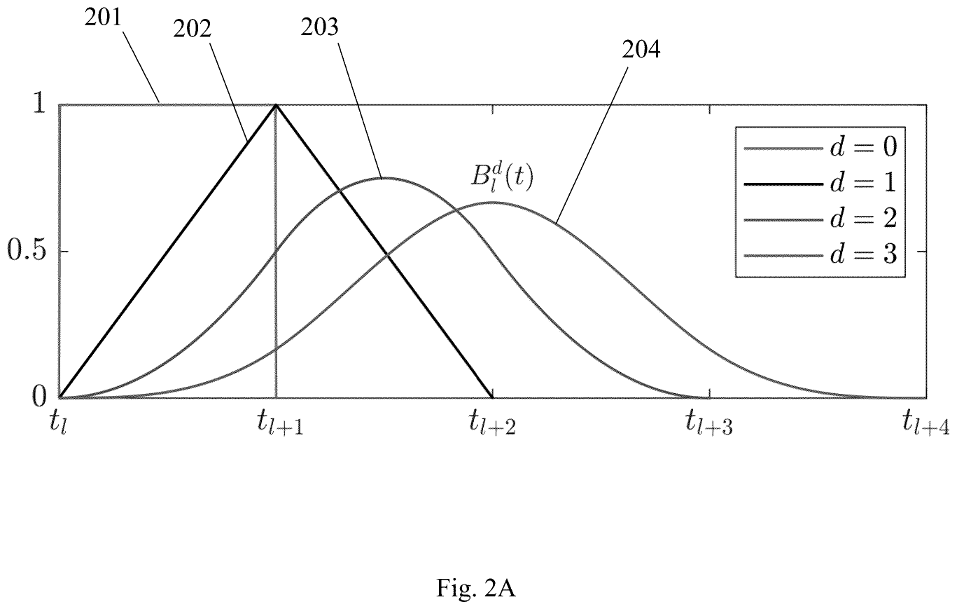

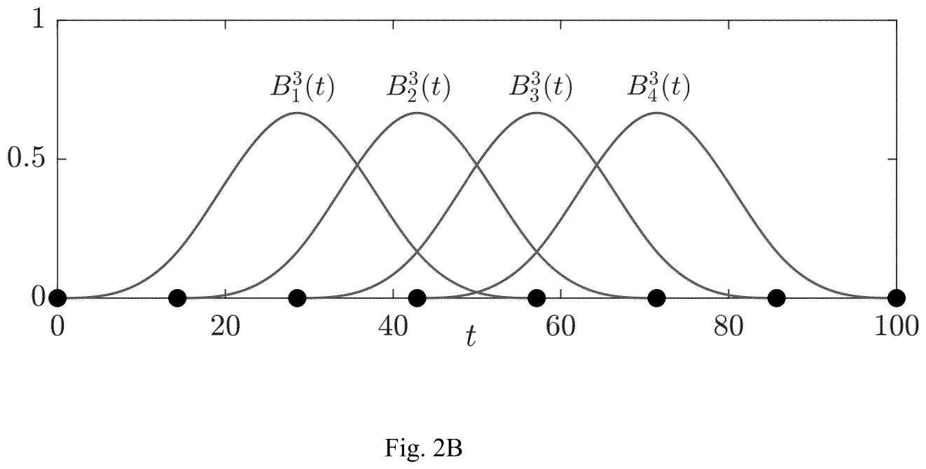

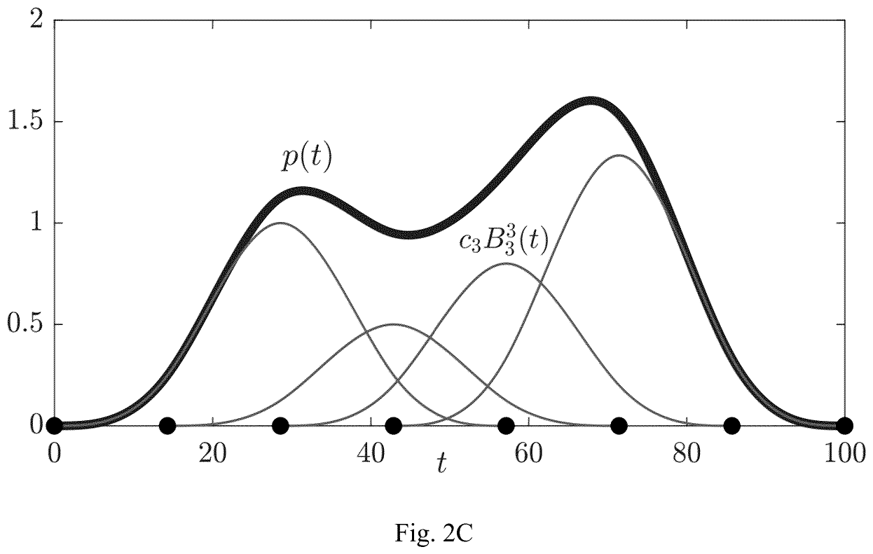

Each Bₗ^{d}(t) has local support in [tₗ, t_{l+d+1}], and, collectively, {Bₗ^{d}(t)}ₗ₌₁^{L} forms a basis of the space of piecewise polynomials of degree d which share the same set of connecting points and are d−1 times continuously differentiable (Höllig & Hörner, Approximation and Modeling with B-Splines. Philadelphia, PA: Society for Industrial and Applied Mathematics, 2013). FIG. 2A, FIG. 2B, and FIG. 2C illustrate several representative B-spline basis functions as well as the use of B-spline basis functions in piecewise polynomial representation. FIG. 2A shows B-spline basis functions Bₗ^{d}(t) of degree d equal to 0 (201), 1 (202), 2 (203), and 3 (204) with support in [tₗ, t_{l+d+1}]. FIG. 2B shows cubic B-spline basis functions defined on eight uniformly spaced knots in the interval [0,100]. Finally, FIG. 2C shows a representation of a piecewise polynomial p(t) as a linear combination of four cubic B-spline basis functions, i.e.,

$$p ( t ) = {\sum }_{l = 1}^{4} {c}_{l} {B}_{l}^{3} ( t ) .$$

Note that B-spline basis functions are controlled by the knot sequence and the degree of the piecewise polynomials.

Given that {αₙ}ₙ₌₁^{N} and {TRₙ}ₙ₌₁^{N} are samples from α(t) and TR(t),

$${α}_{n} = {\sum }_{l = 1}^{{L}_{α}} {g}_{l} {B}_{l}^{{d}_{α}} ( n ) , \qquad Equation 8$$

$$T {R}_{n} = {\sum }_{l = 1}^{{L}_{TR}} {h}_{l} {B}_{l}^{{d}_{TR}} ( n ) \qquad Equation 9$$

for n=1, . . . , N. In matrix-vector form, Eq. [8] and Eq. [9] can be rewritten as

$${U}_{α} = {B}_{α} g \qquad Equation 10$$

$${U}_{TR} = {B}_{TR} h \qquad Equation 11$$

where B_{α}∈ [symbol] ^{N×L}^{_{α}} denotes the B-spline coefficient matrix for FA with [B_{α}]_{n,l}=Bₗ^{d}^{_{α}}(n), B_{TR} ∈ [symbol] ^{N×L}^{_{TR}} denotes the B-spline coefficient matrix for TR with [B_{TR}]_{n,l}=Bₗ^{d}^{_{TR}}(n), and g∈ [symbol] ^{L}^{_{α}} and h∈ [symbol] ^{L}^{_{TR}} contain the B-spline coefficients for the FA and TR sequences, respectively.

Note that Equation 10 and Equation 11 enforce the subspace constraints for the acquisition parameter sequences, and that the two subspaces spanned by the columns of B_{α} and B_{TR} are controlled by the degrees and the knot sequences of the corresponding B-spline basis functions. According to the Schoenberg-Whitney condition (Höllig & Hörner, Approximation and Modeling with B-Splines. Philadelphia, PA: Society for Industrial and Applied Mathematics, 2013), the dimensions of the two subspaces are L_{α} and L_{TR}, which in practice can be chosen to be much smaller than N to represent the desired acquisition sequences. In some embodiments, the dimensions used for the acquisition parameter sequences disclosed herein may be 5, 10, 20, 25, 50, 100, or any other suitable dimension. Integrating the subspace constraints with the upper and lower bounds on the FAs and TRs, the proposed constraint set can be written as

$$𝒰 = \{ g ∈ {ℝ}^{{L}_{α}} , h ∈ {ℝ}^{{L}_{T R}} \vert {α}^{min} ≼ {B}_{α} g ≼ {α}^{max} , T {R}^{min} ≼ {B}_{TR} h ≼ T {R}^{max} . \} . \qquad Equation 12$$

With the low-dimensional B-spline subspace constraints, [symbol] can provide a much smaller search space than [symbol] in Equation 3, while respecting the underlying physical constraints. This method of calculating the acquisition sequences greatly improves the computational efficiency of solving the OED problem using numerical algorithms.

In various embodiments, different degrees of B-spline basis functions may be chosen to construct piecewise polynomial representations of time series functions. In some embodiments, the degree may be chosen manually, for example based on prior knowledge of existing time series functions and the typical shapes those functions take—for example, if a particular time series was a binary sequence of square waves oscillating between two values, a degree of 0 may be chosen. In another example, if a particular time series is typically a sawtooth wave with linear progressions between two or more values, a degree of 1 may be used, and so on. In some embodiments, the ideal degree for a given parameter may be algorithmically chosen, for example using a machine learning algorithm or via an algorithmic function of a number of inputs, including but not limited to parameters related to the subject(s) being imaged, the devices being used in the imaging, and/or the target tissue(s) of the imaging.

Certain embodiments disclosed herein include the calculation of improved time series functions, for example stimulus patterns over a fixed time sequence, using a set of basis functions and accompanying coefficients, for example B-spline basis functions with magnitude/damping/gain coefficients and knot/phase coefficients. It is understood that in other contemplated embodiments, other basis functions may be used to calculate improved time series, including but not limited to wavelets, radial basis, Fourier basis, or any other set of basis functions.

Combining the equations disclosed above, the OED problem can be formulated as follows:

$$\begin{matrix}{max}_{g , h} & - {\sum }_{l = 1}^{L} tr ( {W}^{( l )} {C}^{1 / 2} ( {θ}^{( l )} , [ {B}_{α} g , {B}_{TR} h ] ) ) \\ s . t . & {α}^{min} ≼ {B}_{α} g ≼ {α}^{max} , T {R}^{min} ≼ {B}_{TR} h ≼ T {R}^{max} .\end{matrix} \qquad Equation 13$$

Solutions to Equation 13 can be obtained using a number of algorithms, such as nonlinear programming (Nocedal & Wright, Numerical Optimization. New York: Springer, 2nd edition, 2006) or stochastic programming methods (Spall J C, Introduction to Stochastic Search and Optimization: Estimation, Simulation, and Control. New York, NY: John Wiley & Sons, 2003). As in (Zhao et al., IEEE Trans Med Imaging 2019; 38:844-861), a state-of-the-art nonlinear programming algorithm is applied, which in some embodiments is sequential quadratic programming (SQP), to solve this problem in Equation 13. The SQP algorithm is a gradient-based iterative algorithm which, at each iteration, solves a constrained quadratic programming problem in which the objective function is a quadratic approximation of the original objective function and the constraint set is the same as Equation 13. Other exemplary algorithms which may be used to solve the equation in Equation 13 include, but are not limited to, other nonlinear programming algorithms (e.g., interior point algorithms) and stochastic programming algorithms (e.g., genetic algorithms and simulated annealing algorithms).

The dominant computational cost of the SQP algorithm for solving Equation 13 is the gradient evaluation, which involves the simulation of a Bloch equation based dynamical system. While there are several methods to calculate the gradient of an objective function (Nocedal & Wright, Numerical Optimization. New York: Springer, 2nd edition, 2006), e.g., finite differencing and automatic differentiation, an OED problem with a reduced search space generally enables more efficient gradient calculation. For example, the computational complexity of gradient calculation via finite differences is linear with respect to the dimension of the search space in Equation 13, which, in theory, would provide a factor of 2N/(L_{α}+L_{TR}) improvement in computational efficiency, as compared to the OED without the subspace constraints.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the system and method of the present invention. The following working examples therefore, specifically point out the exemplary embodiments of the present invention, and are not to be construed as limiting in any way the remainder of the disclosure.

### Example #1—Solution Algorithm

### Methods

First, the performance of the disclosed approach in optimizing a representative MR Fingerprinting pulse sequence was evaluated, i.e., the inversion recovery fast imaging with steady state precession (IR-FISP) sequence (Jiang et al., Magn Reson Med 2015; 74:1621-1631), for neuroimaging. The disclosed approach was compared with the state-of-the-art methods in (Zhao et al., IEEE Trans Med Imaging 2019; 38:844-861) using numerical simulations, phantom experiments, and in vivo experiments. The related details are provided in the following subsections.

### Implementation Details

The implementation of the disclosed approach is described below. First, the tissue parameters {θ^{(l)}}ₗ₌₁^{L} were specified for the design metric in Equation 13. Three representative tissues relevant to neuroimaging applications were selected for the experimental design, i.e., θ⁽¹⁾=[700 ms, 60 ms, 0.6]^{T}, θ⁽²⁾=[850 ms, 80 ms, 0.6]^{T}, and θ⁽³⁾=[1100 ms, 102 ms, 0.6]^{T}. Furthermore, tissue relaxation times (i.e., T₁ and T₂) were assumed to be of primary interest, and so [ω₁, ω₂, ω₃]=[3.45,1,1.6] were manually chosen for the weighting matrix W^{(l)} with diagonal entries W_{p,p}^{(l)}=ωₚ/θₚ^{(l)} to ensure that the optimized pulse sequence achieved good performance for different tissue components of interest.

The bound constraints for the design acquisition sequences were then specified. For the FA sequence, the lower bounds: αₙ^{min}=10° for n=1, . . . , N, and upper bounds

$${α}_{n}^{max} = \{ \begin{matrix}180 ° , & if n = 1 , \\ 60 ° , & if 2 \leq n \leq N .\end{matrix}$$

were imposed. Here, the upper bound on the first flip angle allowed the use of a 180° inversion pulse, which is often advantageous to the T₁ estimation. Moreover, the upper bounds and lower bounds for the TR sequence were set as TRₙ^{min}=11 ms and TRₙ^{max}=15 ms for 1≤n≤N. Note that the above bound constraints are consistent with the range of FAs and TRs in conventional MR Fingerprinting experiments (Jiang et al., Magn Reson Med 2015; 74:1621-1631).

The B-spline subspace constraints were also specified. These constraints, which enforce the desired structure of the FA and TR sequences, are controlled by the degrees and knot sequences of the B-spline basis functions. To exploit the special structure of the optimized FA and TR sequences (Zhao et al., IEEE Trans Med Imaging 2019; 38:844-861), in the present disclosure, the respective degrees of the B-spline basis functions were set as d_{α}=3 and d_{TR}=0. Moreover, uniformly spaced knots in the interval [1, N] were used for the FA and TR sequences such that the dimensions of the two subspaces are L_{α}=L_{TR}=N/20. Here repeated knots were used at the boundary of the interval, which ensured that the B-spline basis functions formed a complete basis for the subspaces. Additionally, the first flip angle was excluded from the spline representation to allow a potential discontinuity from an inversion pulse to the subsequent RF pulse. As described in the Results section below, the above B-spline parameters consistently yielded good empirical results for the OED problem. Further discussions on the parameter selection are included below in the Discussion section.

The SQP algorithm was applied to solve the OED problem in Equation 13. The SQP algorithm requires the gradient evaluation at each iteration, for which a finite difference approximation was used. This requires a large number of objective function evaluations at each iteration, which involves extensive Bloch simulations. In some embodiments, the number of evaluations required is on the order of 2N, where N is the acquisition length, i.e., the number of RF pulses applied. In general, the cost is on the order of N*D, where D is the number of optimizable acquisition parameter sequences.

Bloch simulations were performed using the summation-of-isochromats approach (Zhao et al., IEEE Trans Med Imaging 2019; 38:844-861; Maidens et al., Parallel dynamic programming for optimal experiment design in nonlinear systems. In: Proc. IEEE Conf. Decis. Control, 2016. pp. 2894-2899; Malik et al., Equivalence of EPG and isochromat-based simulation of MR signals. In: Proc. Int. Symp. Magn. Reson. Med., 2016. p. 3196) with a total number of 400 isochromats. The SQP algorithm was initialized by performing a least-squares fit of the B-spline coefficients to the FA and TR sequences in the conventional MR Fingerprinting experiments (Jiang et al., Magn Reson Med 2015; 74:1621-1631), and the algorithm was terminated when the norm of the gradient was less than the pre-specified tolerance ϵ=10⁻⁶ or the maximum number of iterations N_{Iter}=4×10⁴ was reached. In some embodiments, the maximum iteration is chosen as a number large enough to comfortably reach the selected pre-specified tolerance, while also restricting the algorithmic runtime to a reasonable duration in case the algorithm fails to converge. Finally, the algorithm was implemented on a Linux server with dual Intel Xeon Gold 6248R processors (each with 3.00 GHz and 24 cores) and 1.50 TB RAM running MATLAB® R2020a. Parallel computing was used for the gradient evaluation across all 48 cores.

### Method Comparison

The disclosed approach was compared with the two state-of-the-art methods described in (Zhao et al., IEEE Trans Med Imaging 2019; 38:844-861). The first method, referred to herein as Optimized-I, formulates the OED problem as follows:

$$\begin{matrix}{max}_{{U}_{α} , {U}_{TR}} & - {\sum }_{l = 1}^{L} tr ( {W}^{( l )} {C}^{1 / 2} ( {θ}^{( l )} , [ {U}_{α} , {U}_{TR} ] ) ) \\ s . t . & {α}^{min} ≼ {U}_{α} ≼ {α}^{max} , T {R}^{min} ≼ {U}_{TR} ≼ T {R}^{max} .\end{matrix} \qquad Equation 15$$

Note that this method optimizes the same objective function as the disclosed approach, except it only imposes the bound constraints on the FAs and TRs.

It has been demonstrated that constraining the flip angle variation in the experimental design is often beneficial for parameter decoding from highly-undersampled MR Fingerprinting experiments (Zhao et al., IEEE Trans Med Imaging 2019; 38:844-861). This leads to the following OED formulation:

$$\begin{matrix}{max}_{{U}_{α} , {U}_{TR}} & - {\sum }_{l = 1}^{L} tr ( {W}^{( l )} {C}^{1 / 2} ( {θ}^{( l )} , [ {U}_{α} , {U}_{TR} ] ) ) \\ s . t . & {α}^{min} ≼ {U}_{α} ≼ {α}^{max} , T {R}^{min} ≼ {U}_{TR} ≼ T {R}^{max} , \vert D {U}_{α} \vert ≼ Δ {α}_{n}^{max} ,\end{matrix} \qquad Equation 16$$

where the maximum FA variation is set as Δαₙ^{max}=1° as in (Zhao et al., IEEE Trans Med Imaging 2019; 38:844-861). This approach is referred to herein as Optimized-II. It is clear that Optimized-II also uses the same objective function as the disclosed approach, but with a different constraint set, i.e., [symbol] in Equation 3. Here, by constraining the flip angle variation, Optimized-II reduces the highly oscillatory behavior of the magnetization evolutions, which has been shown to improve the accuracy of parameter estimation (Zhao et al., IEEE Trans Med Imaging 2019; 38:844-861). As disclosed in the Results section below, the disclosed approach provides similar benefits as Optimized-II, but uses completely different constraints, i.e., the B-spline based subspace constraints, which results in a much smaller search space for the OED problem.

The disclosed approach was compared with Optimized-I and Optimized-II in terms of computational efficiency and estimation accuracy. The same algorithm was used (i.e., SQP) as the disclosed method to solve Optimized-I and Optimized-II with an identical algorithmic setup (e.g., initialization and stopping criteria). The three approaches were evaluated on the same computing platform as described above.

### Numerical Simulations

Numerical simulations were conducted to evaluate the disclosed approach. Specifically, a numerical brain phantom was constructed using T₁, T₂, and spin density maps from the Brainweb database (Collins et al., Design and construction of a realistic digital brain phantom. IEEE Trans Med Imaging 1998; 17:463-468), which models a single-channel MR Fingerprinting experiment. The field-of-view (FOV) was set as 300×300 mm² and the matrix size as 256×256. he IR-FISP sequence was simulated to generate contrast-weighted images with the acquisition parameters from conventional experiments (Jiang et al., Magn Reson Med 2015; 74:1621-1631), Optimized-I, Optimized-II, and the disclosed approach at two acquisition lengths: N=400 and 600.

The undersampled k-space data was synthesized from the contrast-weighted images using a nonuniform Fourier transform (Fessler & Sutton, Nonuniform fast Fourier transforms using min-max interpolation. IEEE Trans Signal Process 2003; 51:560-574). As in (Jiang et al., Magn Reson Med 2015; 74:1621-1631), one spiral interleaf was acquired (out of the full set of 48 interleaves) per each TR in the simulation. Complex-valued white Gaussian noise was added to the measured k-space data with a pre-specified signal-to-noise ratio level. Here the signal-to-noise ratio was defined as SNR=20 log₁₀(s/σ), where s denotes the average value of spin density in a region of white matter and σ denotes the noise standard deviation. Specifically, in the disclosed experiment, the numerical simulations were performed at SNR=33 dB. Although various examples presented herein specify an SNR at a given value, for example 33 dB, it is understood that noise variance appears in the algorithm as a global scaling constant, and so does not in some embodiments impact the performance of the experimental design.

The tissue parameter maps were reconstructed from the undersampled k-space data using a maximum likelihood (ML) reconstruction (Zhao et al., Maximum likelihood reconstruction for magnetic resonance fingerprinting. IEEE Trans Med Imaging 2016; 35:1812-1823), in which a dictionary for the magnetization evolutions was generated with T₁ and T₂ values relevant to neuroimaging. Specifically, the value of T₁ was in the range [20,3000] ms, with an increment of 10 ms for [20,1500] ms and an increment of 30 ms for [1501,3000] ms. The value of T₂ was in the range [30,500] ms, with an increment of 1 ms for [30,200] ms and an increment of 5 ms for [201,500] ms.

The reconstruction accuracy was evaluated using the following two metrics: (a) the overall relative error, i.e., ∥I−Î∥₂/∥I∥₂, where I and Î respectively denote the ground truth and reconstructed tissue parameter maps, and (b) the voxelwise relative error, i.e., |Iᵥ−Îᵥ|/|Iᵥ|, where Iᵥ and Îᵥ are the values of the ground truth and reconstructed tissue parameter maps at the vth voxel, respectively. The bias and variance of the reconstructed tissue parameter maps were evaluated using Monte Carlo (MC) simulations. Specifically, MC simulations were constructed with 100 trials and evaluating the normalized bias:

$${Nbias}_{v} = \hat{𝔼} [ \vert {I}_{v} - {\hat{I}}_{v} \vert ] / \vert {I}_{v} \vert \qquad Equation 17$$

and the normalized standard deviation:

$${Nstd}_{v} = \sqrt{\hat{𝔼} [ {\vert {I}_{v} - \hat{𝔼} [ {\hat{I}}_{v} ] \vert}^{2} ]} / \vert {I}_{v} \vert , \qquad Equation 18$$

where [symbol] [·] denotes the sample mean over the MC trials.

### Phantom Experiments

Phantom experiments were performed to evaluate the disclosed approach. Specifically, a physical phantom was used that consisted of 12 tubes filled with solutions of gadolinium and agar at different concentrations, creating different combinations of T₁ and T₂ values relevant to neuroimaging. The experiments were conducted on a 3T Siemens Magnetom Prisma scanner (Siemens Healthineers, Erlangen, Germany) equipped with a 20-channel head receiver coil. Here the acquisition parameters from the conventional experiment were used (Jiang et al., Magn Reson Med 2015; 74:1621-1631), Optimized-I, Optimized-II, and the disclosed approach at acquisition length N=400. Other relevant imaging parameters included FOV=300×300 mm², matrix size=256×256, and slice thickness=5 mm.

The MR Fingerprinting experiments were carried out using the same set of spiral trajectories as in (Jiang et al., Magn Reson Med 2015; 74:1621-1631), along with a pilot scan using the vendor-provided gradient echo sequence for the coil sensitivity map estimation. The tissue parameter maps were reconstructed using ML reconstruction (Zhao et al., Maximum likelihood reconstruction for magnetic resonance fingerprinting. IEEE Trans Med Imaging 2016; 35:1812-1823) with a slice-profile corrected dictionary (Ma et al., Slice profile and B_1 corrections in 2D magnetic resonance fingerprinting. Magn Reson Med 2017; 78:1781-1789) containing the same T₁ and T₂ resolutions as in the numerical simulations. To assess the estimation performance associated with different experimental designs, the imaging experiments were repeated 15 times for each acquisition scheme and the voxelwise normalized standard deviation of the reconstructed tissue parameters was evaluated. Note that the above procedure is often employed to evaluate the variance of tissue parameters in experimental studies for quantitative MRI (Zhao et al., IEEE Trans Med Imaging 2019; 38:844-861; Nataraj et al., Optimizing MR scan design for model-based T1, T2 estimation from steady-state sequences. IEEE Trans Med Imaging 2017; 36:467-477).

### In Vivo Experiments

In vivo experiments were conducted to further evaluate the disclosed approach. Two healthy subjects were recruited with the approval of the local Institutional Review Board and informed consent obtained from both subjects. The imaging experiments were conducted on the same MR scanner and with the same set of imaging parameters as the phantom experiments. The conventional, Optimized-I, Optimized-II, and disclosed approaches were evaluated at acquisition length N=400, and each experiment was repeated 15 times. The ML reconstruction was performed and the voxelwise normalized standard deviation of the reconstructed parameter maps were calculated as in the phantom experiments.

### Results

Representative results are shown to illustrate the performance of the disclosed approach. First, the superior computational efficiency of the disclosed approach was demonstrated. Specifically, the MR Fingerprinting experiments were calculated using Optimized-I, Optimized-II, and the disclosed approach at acquisition lengths N=300,400,500, and 600, and the algorithm runtimes recorded below in Table 1. Note that the above approaches optimize the same objective function but with different constraint sets. As can be seen, with a reduced search space enabled by the subspace constraints, the disclosed approach provides a two-order-of-magnitude improvement of the computational efficiency over Optimized-I and Optimized-II. Remarkably, it solves the OED problem in approximately one minute, which significantly enhances the practical utility of the disclosed approach. The times below are recorded in minutes and the acquisition lengths are the number of RF pulses applied during the imaging experiment.

TABLE 1

| Acquisition Length | 300 | 400 | 500 | 600 |
|---|---|---|---|---|
| Optimized-I | 21.16 | 61.43 | 138.34 | 172.47 |
| Optimized-II | 18 | 35.12 | 156.05 | 257.44 |
| Disclosed | 0.44 | 0.62 | 0.73 | 1.17 |

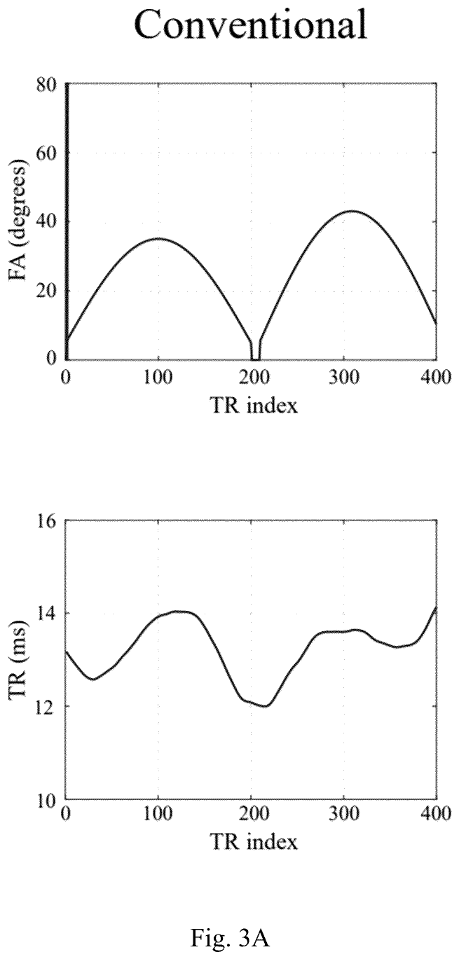

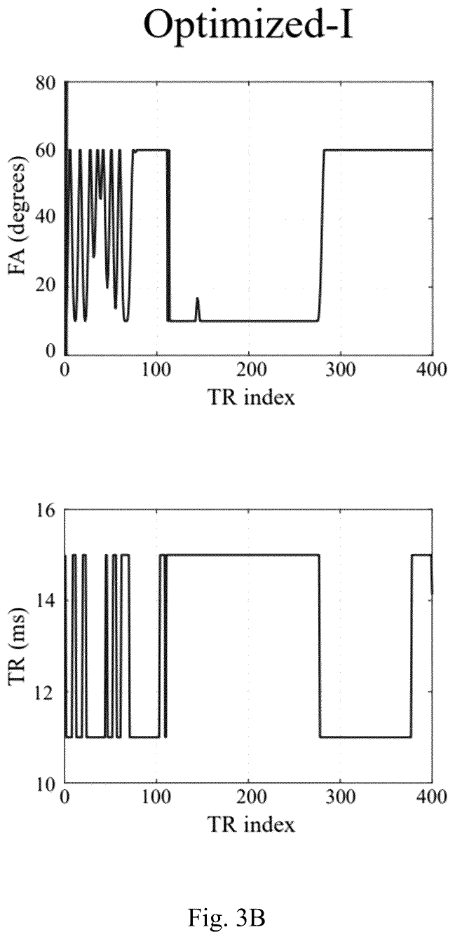

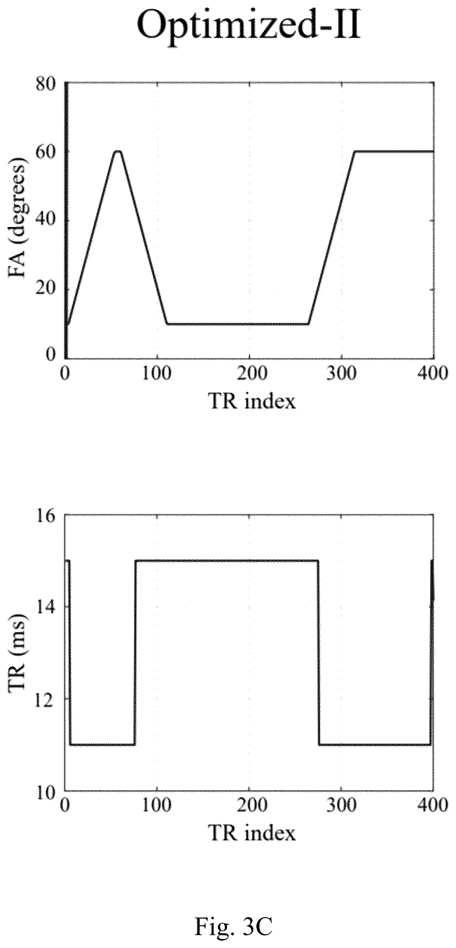

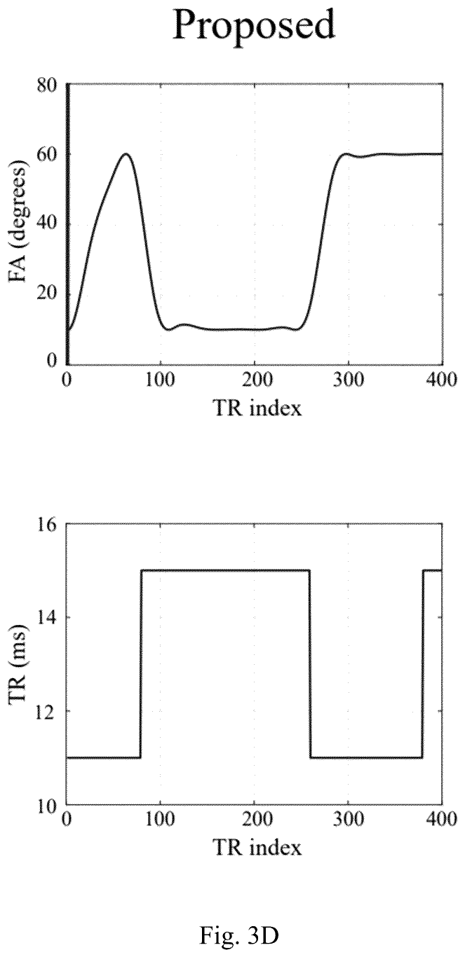

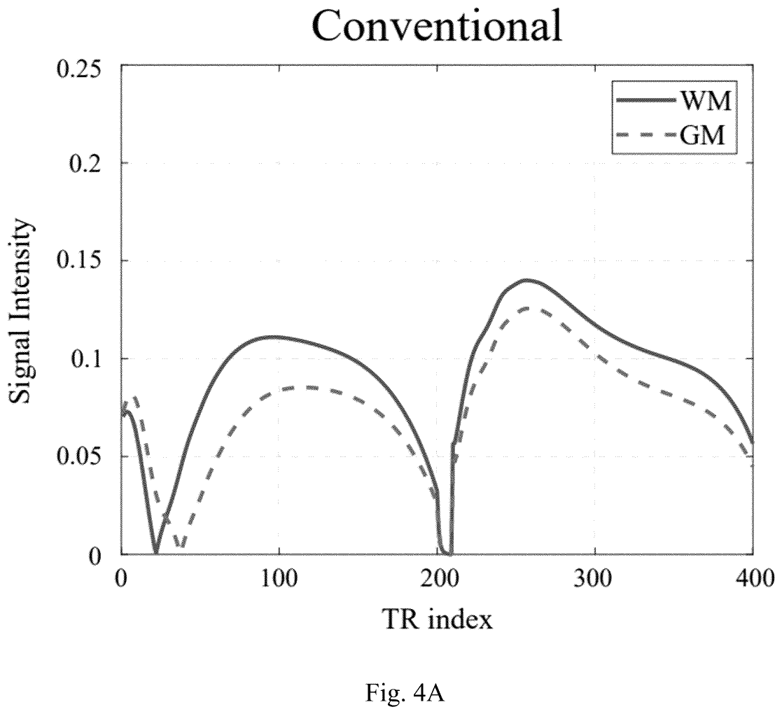

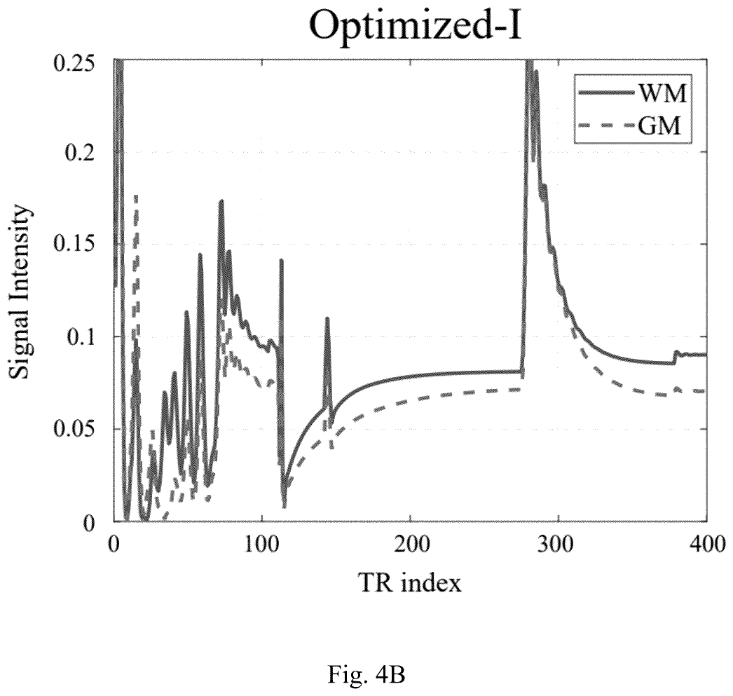

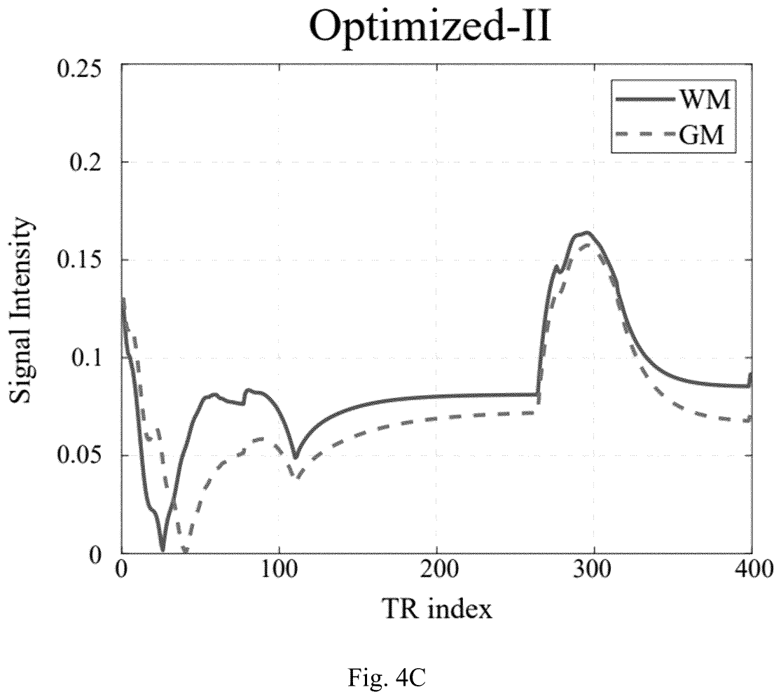

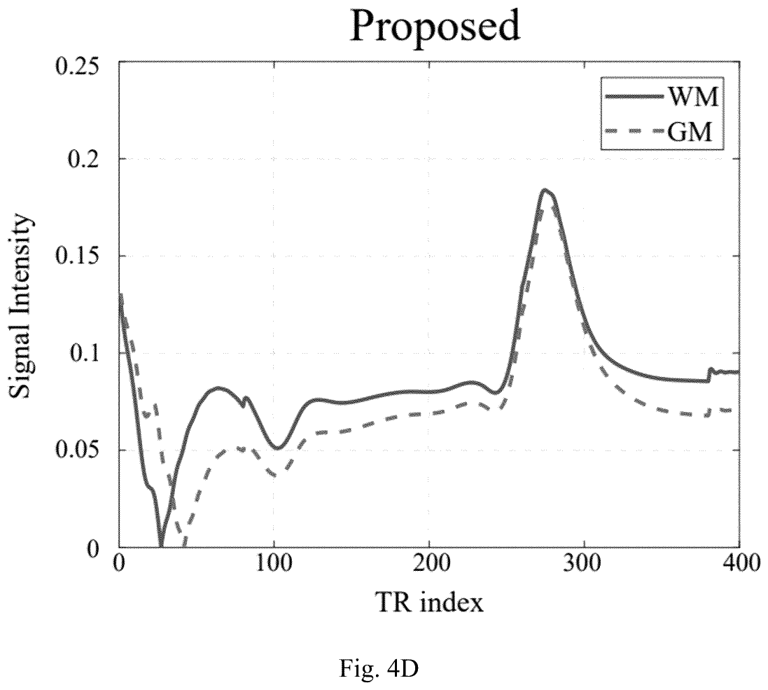

FIG. 3A, FIG. 3B, FIG. 3C, and FIG. 3D show the optimized FA and TR sequences using the above approaches at N=400, with the corresponding magnetization evolutions shown in FIG. 4A, FIG. 4B, FIG. 4C, and FIG. 4D. For all the acquisition schemes, the first RF pulses were 180°, which exceeded the scales of the vertical axes. The total acquisition times for the conventional, Optimized-I, Optimized-II, and disclosed approaches are 5.30 sec, 5.30 sec, 5.25 sec, and 5.22 sec, respectively. As can be seen, with the subspace constraints that enforce the desired structure, the disclosed approach produces very similar FA and TR sequences as Optimized-II. As in the early work (Zhao et al., IEEE Trans Med Imaging 2019; 38:844-861), Optimized-II yields highly structured FA and TR sequences, and by constraining the FA variation, it overcomes the oscillatory behavior of the magnetization evolutions in Optimized-I. With the spline subspace constraints that enforce the desired structure, the disclosed approach results in similar FA and TR sequences and magnetization evolutions as Optimized-II but with significantly better computational efficiency. Similar observations have been obtained at other acquisition lengths.

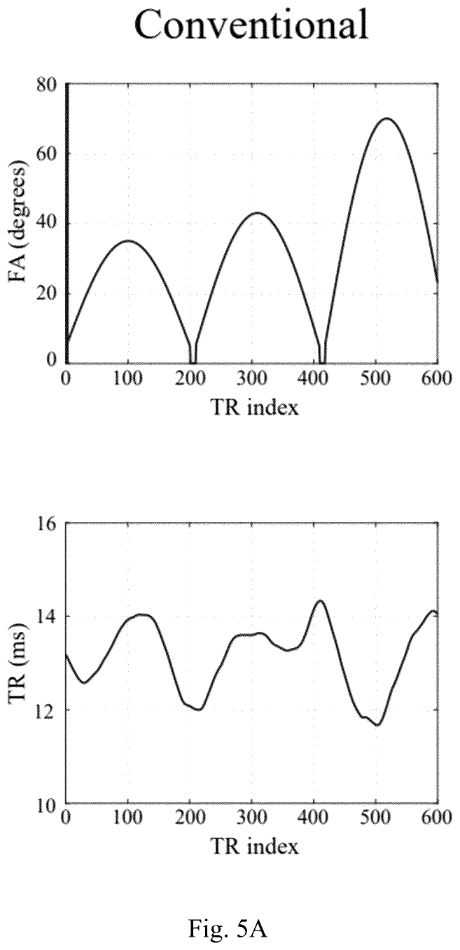

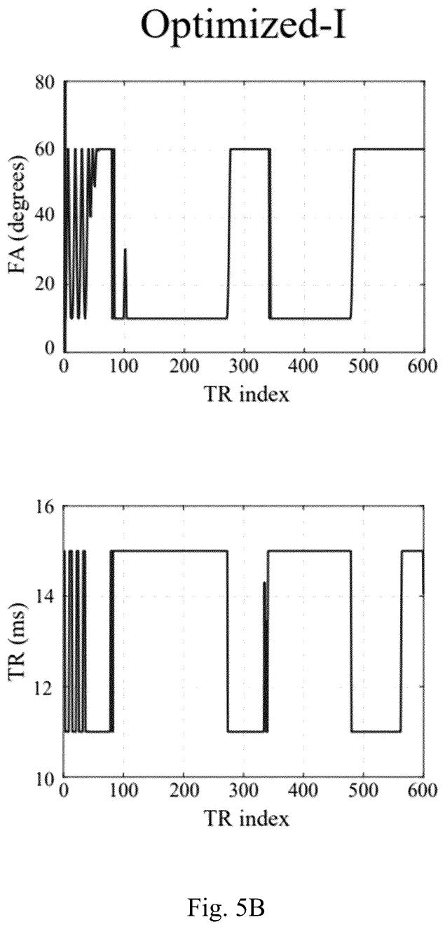

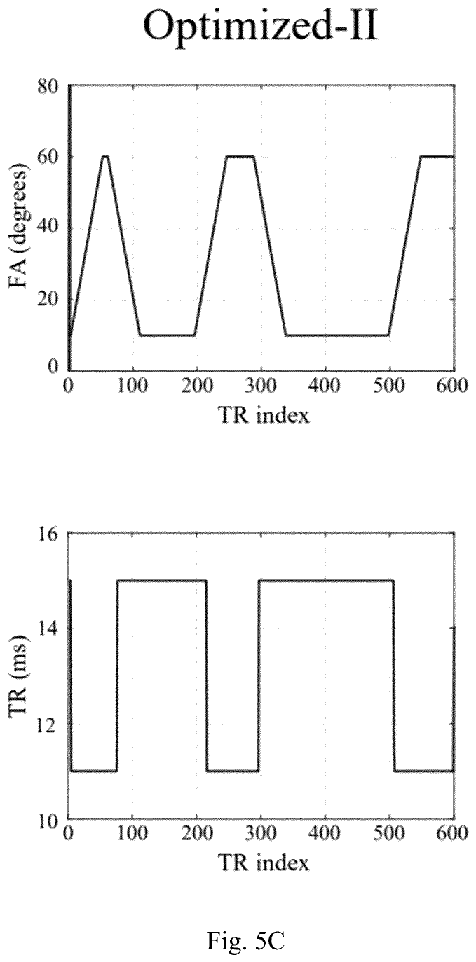

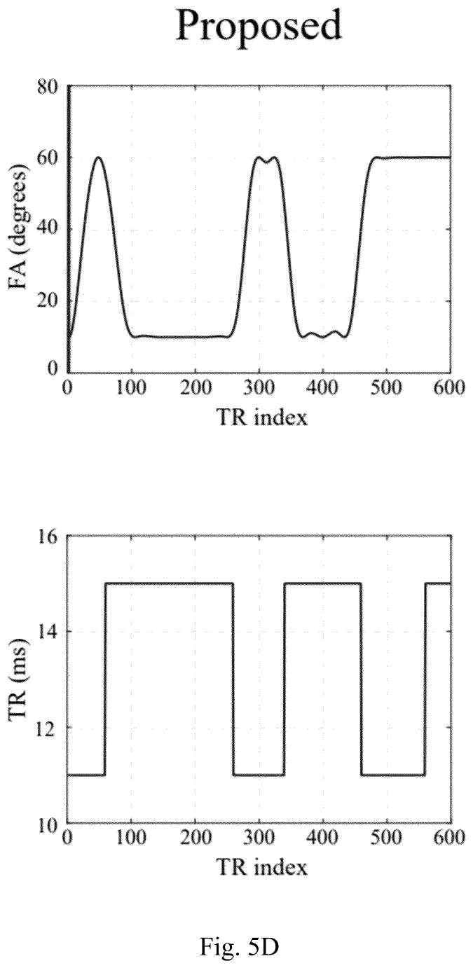

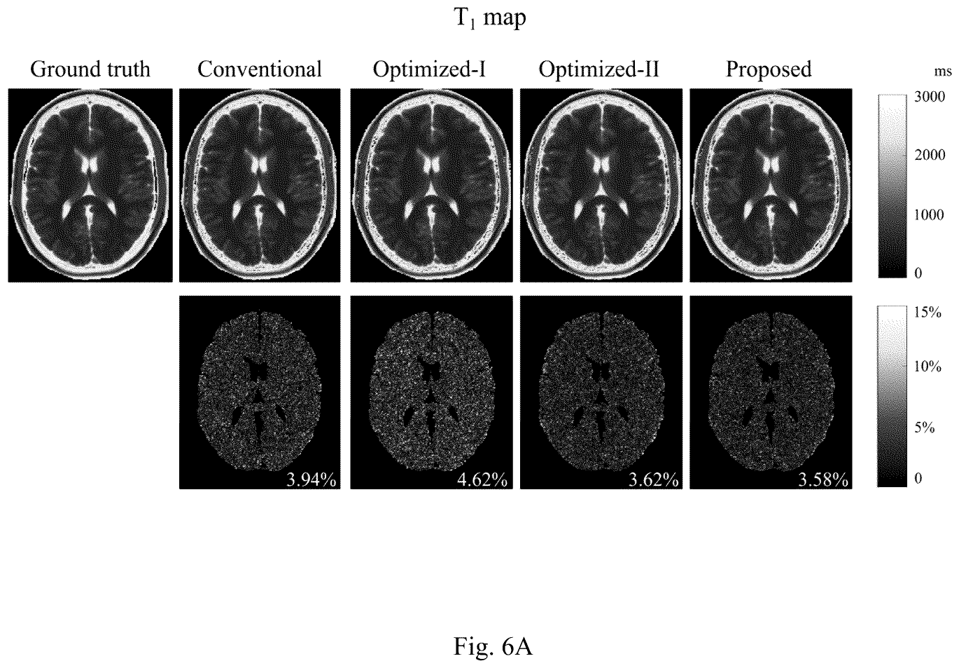

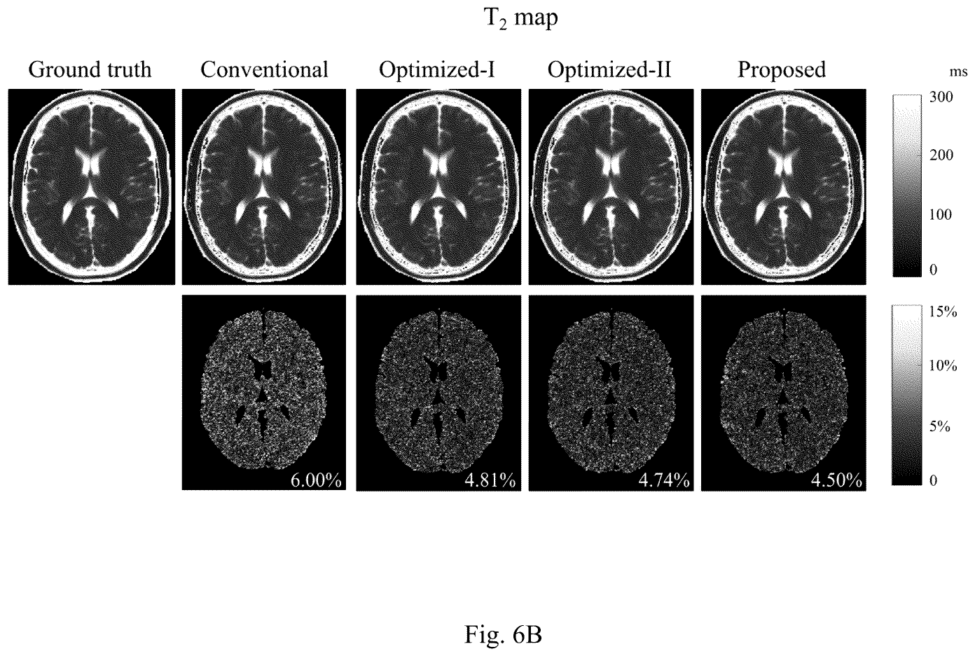

In another example, an acquisition length of N=600 was used. FIG. 5A, FIG. 5B, FIG. 5C, and FIG. 5D show the FA and TR sequences for the conventional (FIG. 5A), Optimized-I (FIG. 5B), Optimized-II (FIG. 5C), and disclosed (FIG. 5D) approaches with N=600. As in the N=400 case (see FIG. 3A, FIG. 3B, FIG. 3C, and FIG. 3D in the main text), the disclosed approach yields optimized acquisition parameters that are similar to those from Optimized-II. FIG. 6A and FIG. 6B show the reconstructed tissue parameter maps from the numerical simulations using the acquisition parameter sequences at N=600 and SNR=33 dB. Along with the reconstructions, the underlying ground truth, associated error maps, and overall relative errors are also shown. As can be seen, the disclosed approach provides improved estimation performance compared to the conventional and Optimized-I approaches and the performance of the disclosed approach is similar to that of Optimized-II.

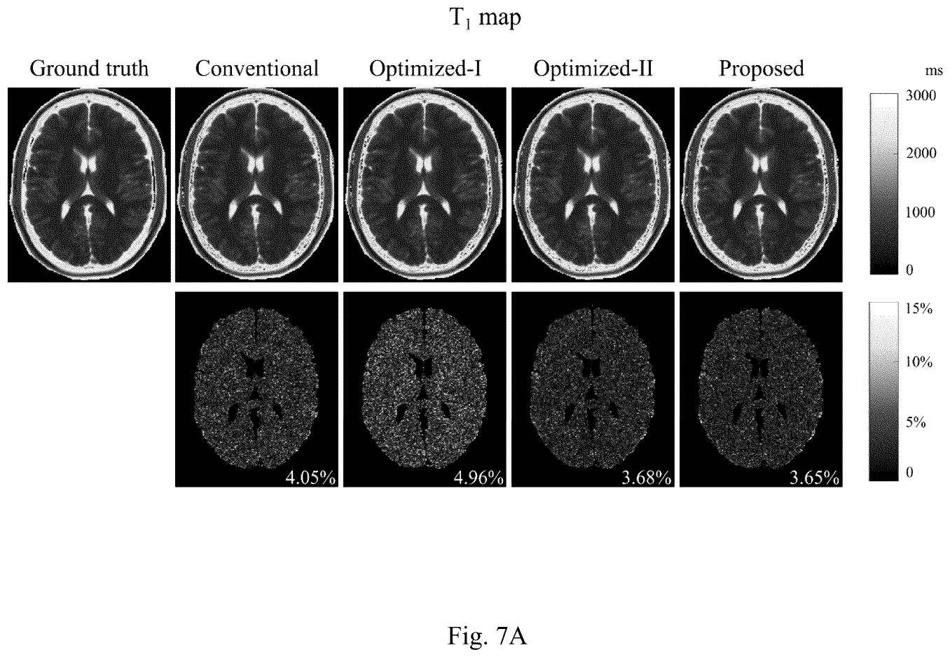

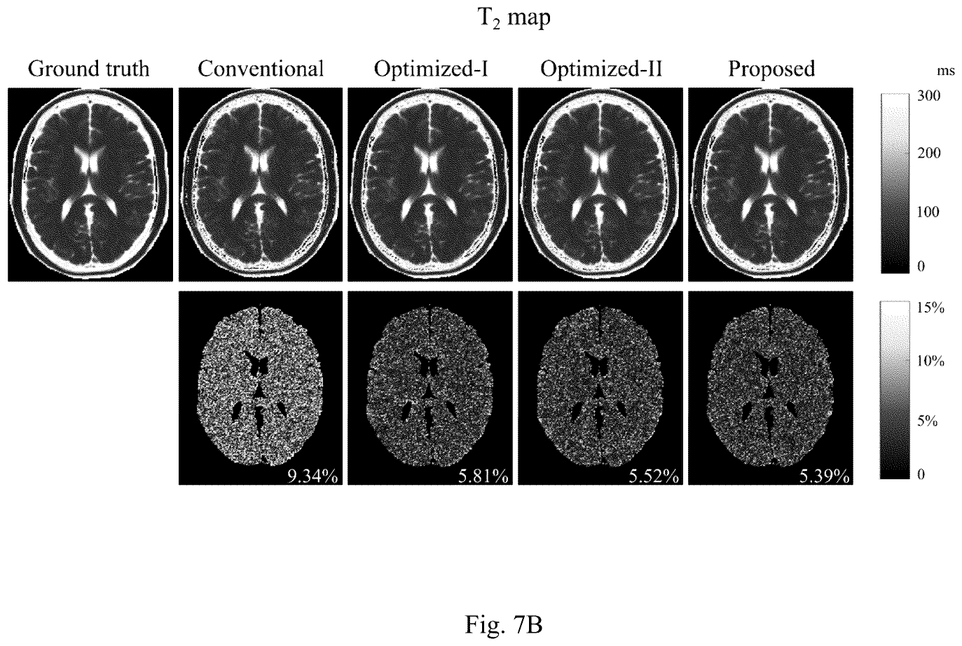

Next, competitive estimation performance of the MR Fingerprinting experiments improved by the disclosed approach is discussed. FIG. 7A and FIG. 7B show the reconstructed T₁ and T₂ maps and the corresponding error maps at N=400 and SNR=33 dB in the simulations. Consistent with the early observation in (Zhao et al., IEEE Trans Med Imaging 2019; 38:844-861), Optimized-II improves the reconstruction accuracy over Optimized-I by controlling the flip angle variation. With the subspace constraints, the disclosed approach provides similar improvement over Optimized-I. Note that the overall relative errors are displayed at the lower right corner of each error map. As can be seen, the disclosed approach provides comparable estimation performance as Optimized-II, while enabling a substantial improvement of the T₂ accuracy and slightly improving the T₁ accuracy over the conventional method. Also note that with smooth magnetization evolutions, both the disclosed approach and Optimized-II offer improvements over Optimized-I.

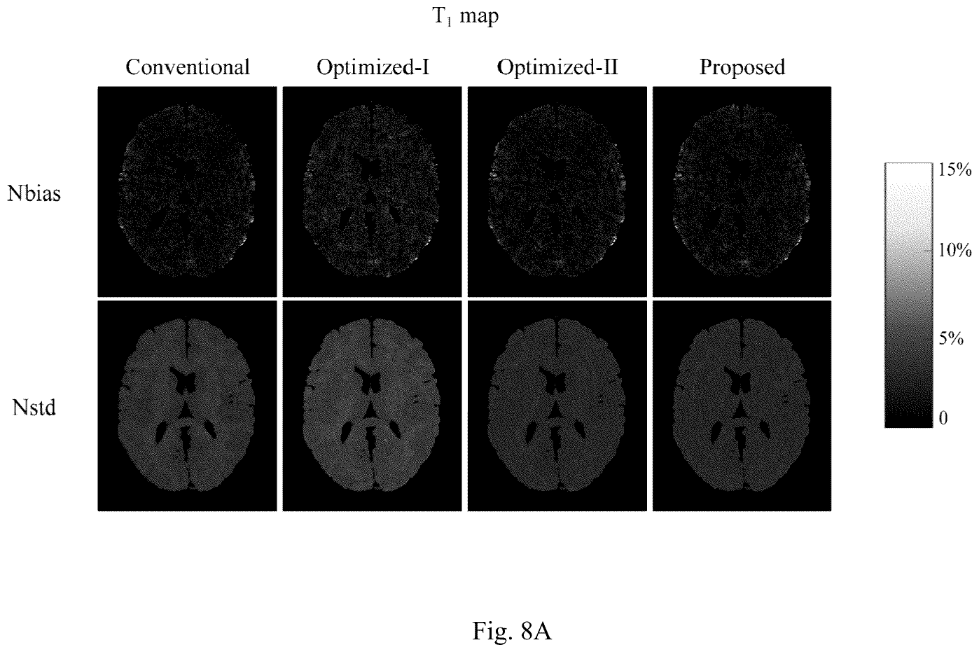

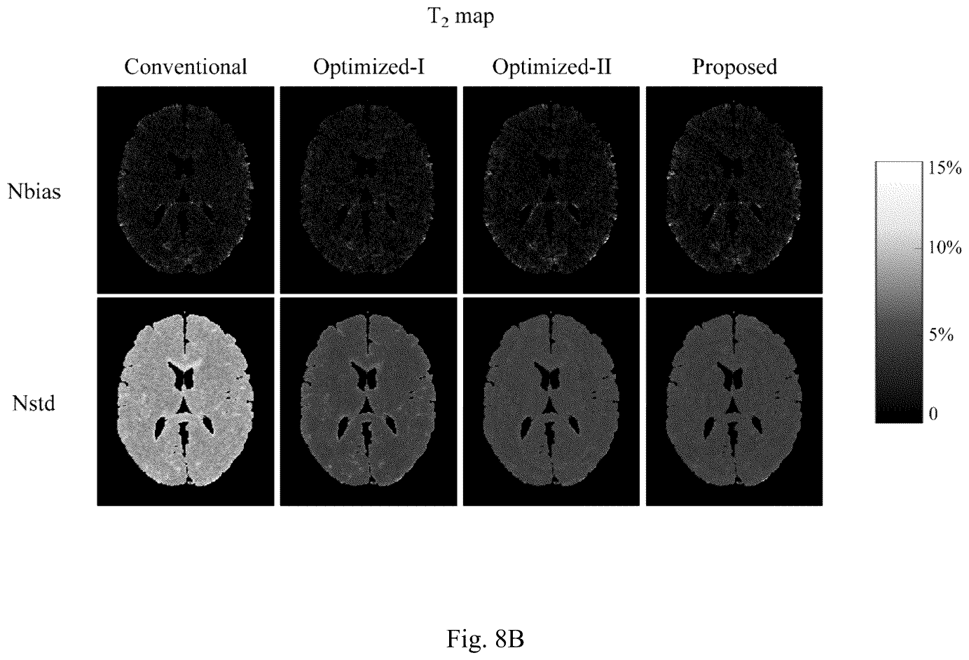

FIG. 8A and FIG. 8B show the bias-variance analysis of the reconstructed MR tissue parameter maps using the acquisition parameters from the conventional experiment, Optimized-I, Optimized-II, and the disclosed approach at N=400 and SNR=33 dB. The normalized biases and standard deviations are shown for the T₁ reconstruction (FIG. 8A) and the T₂ reconstruction (FIG. 8B). The regions associated with the background, skull, scalp, and CSF are set to zero. It is clear that, compared to the conventional experiment (Jiang et al., Magn Reson Med 2015; 74:1621-1631), the disclosed approach reduces the normalized standard deviation of T₂ by a factor of two, while slightly improving that of T₁. With the subspace constraint, it also improves the estimation performance over the unconstrained case, i.e., Optimized-I. Note that the improvement is comparable to that from Optimized-II.

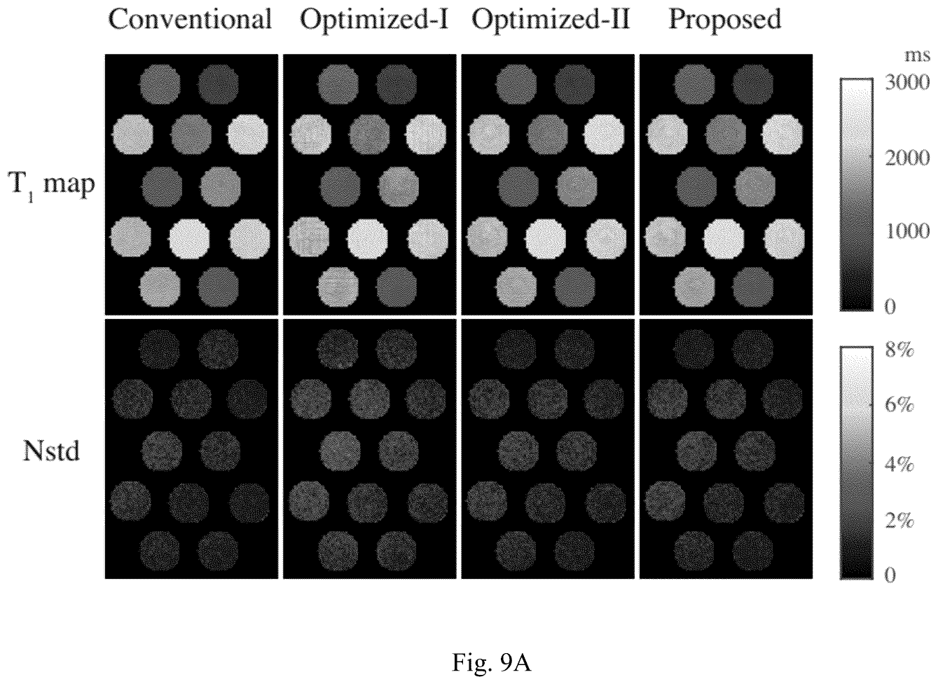

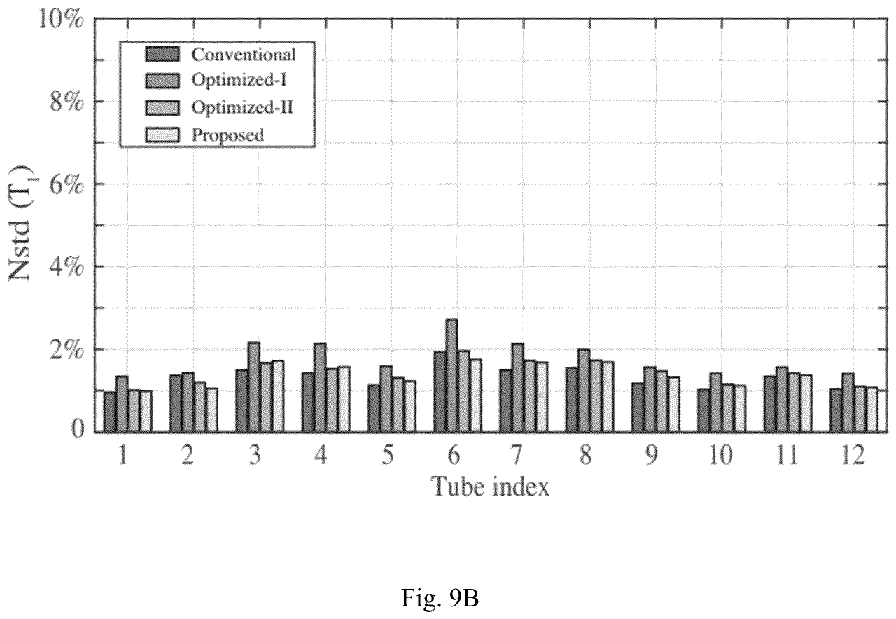

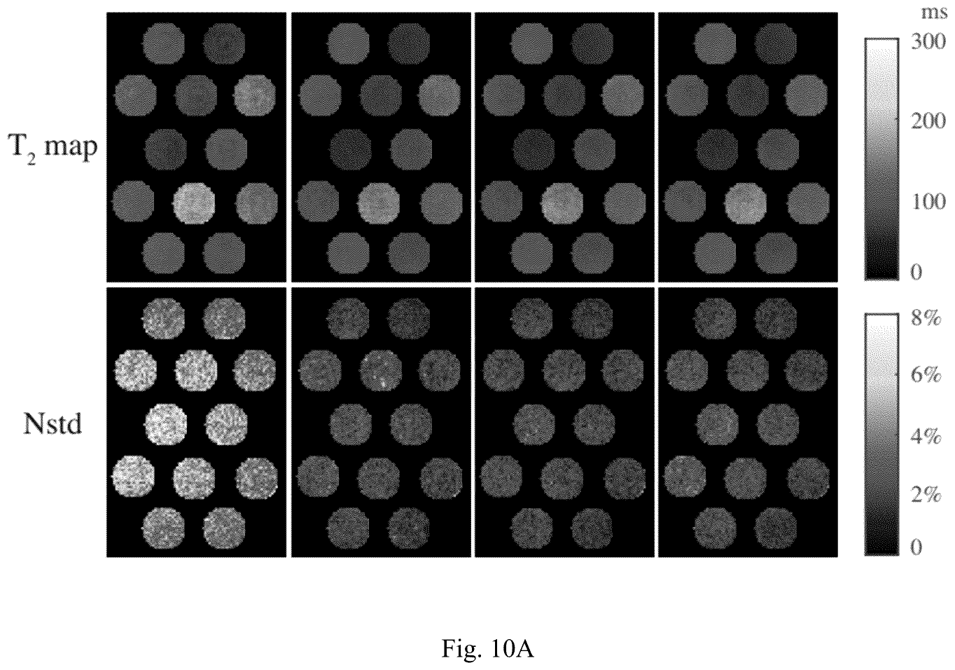

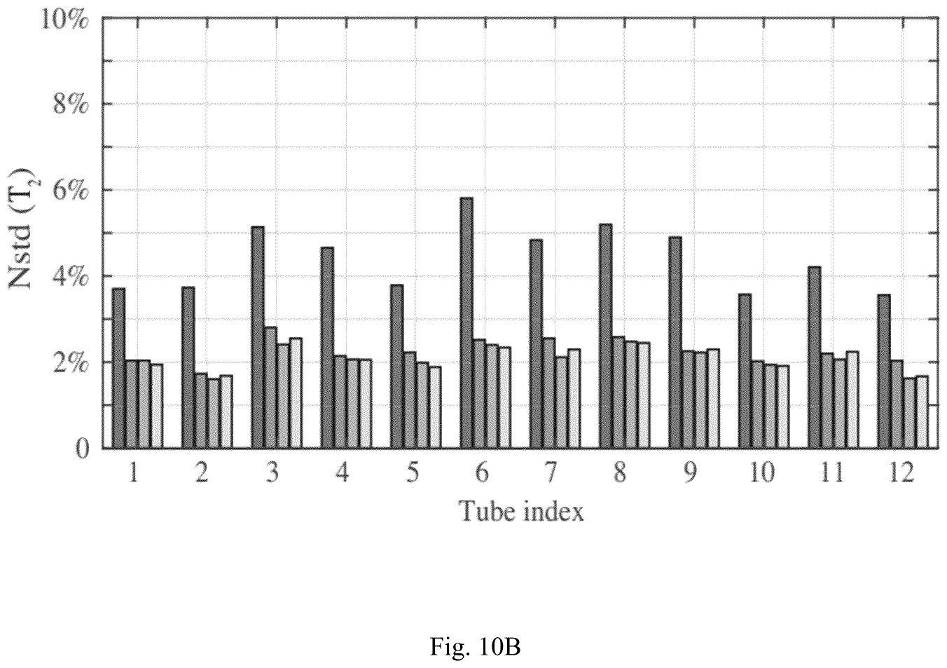

FIG. 9A, FIG. 9B, FIG. 10A, and FIG. 10B show the T₁ and T₂ reconstruction results, respectively, from the phantom experiments at N=400, using the acquisition parameters from the conventional experiment, Optimized-I, Optimized-II, and the disclosed approach. FIG. 9A shows the reconstructed maps and associated normalized standard deviation maps for T₁, and FIG. 9B shoes the normalized standard deviation averaged over each tube. Each of the 12 tubes shows a group of four measurements which are, left to right, the Conventional, Optimized-I, Optimized-II, and disclosed methods. FIG. 10A corresponds to FIG. 9A and FIG. 10B corresponds to FIG. 9B, but FIG. 10A and FIG. 10B show data for T₂.

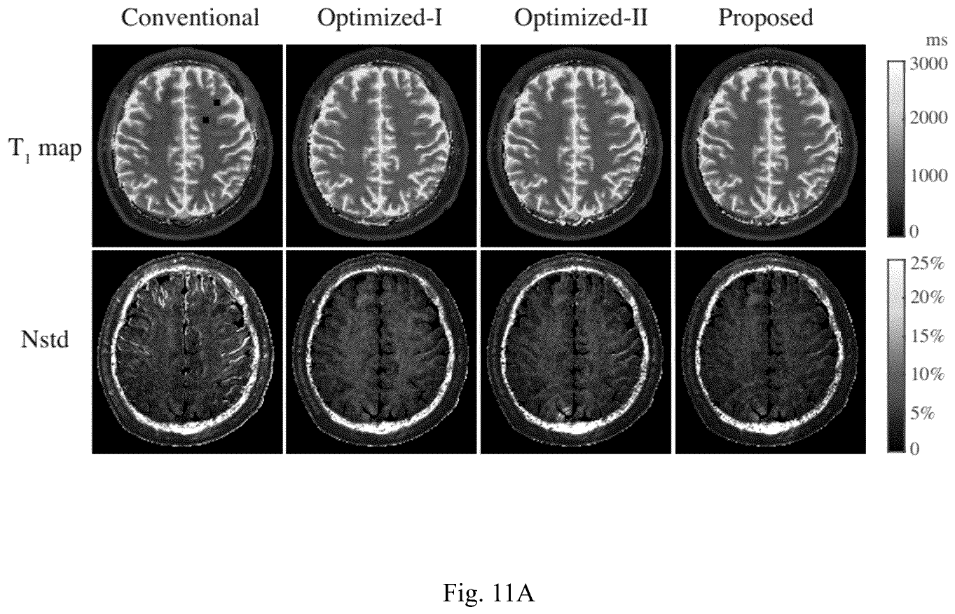

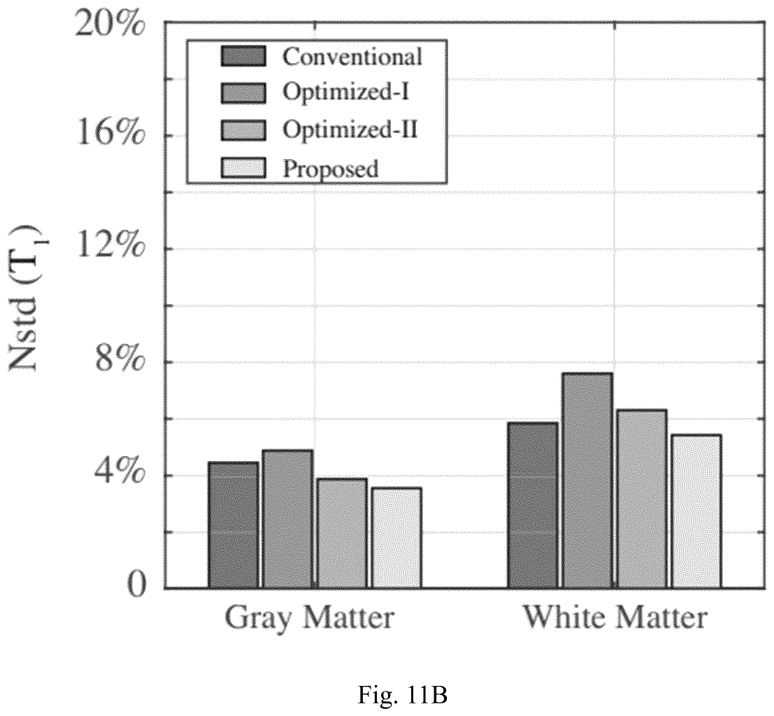

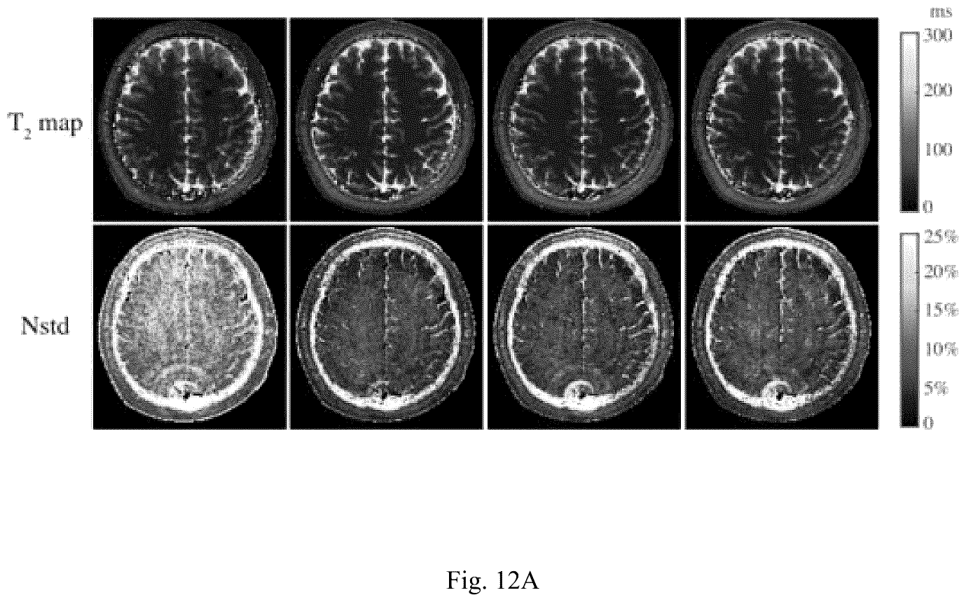

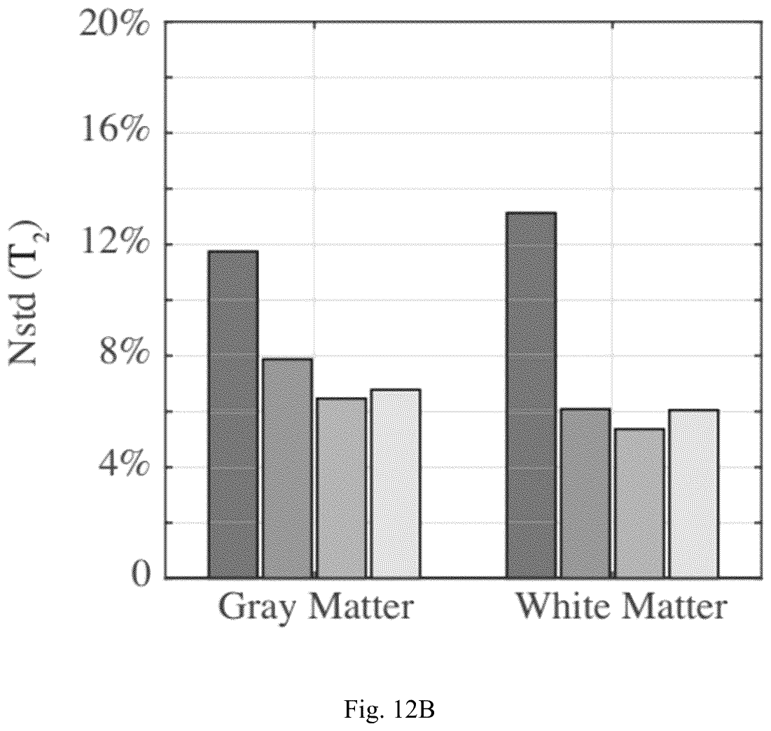

FIG. 11A, FIG. 11B, FIG. 12A, and FIG. 12B show the corresponding results for the in vivo experiments for T₁ (FIG. 11A and FIG. 11B) and 72 (FIG. 12A and FIG. 12B) using the acquisition parameters from the conventional experiment, Optimized-I, Optimized-II, and proposed method. The measurement bars in FIG. 11B and FIG. 12B correspond to the measurement bars in FIG. 9B and FIG. 10B. FIG. 11A and FIG. 12A show reconstructed maps and associated normalized standard deviation maps. FIG. 11B and FIG. 12B show normalized standard deviation averaged over the ROIs in the gray matter and white matter, marked as black squares in 11A. The improvement from the disclosed approach is consistent with the numerical simulations, which further demonstrates that the disclosed approach enables competitive estimation performance while significantly improving the computational efficiency.

### DISCUSSION

The disclosed experimental examples demonstrate that the disclosed approach with the B-spline subspace constraints significantly improves the computational efficiency of solving the OED problem. The use of the B-spline subspace constraints was originally motivated from the structure observed in solving the OED problem (Zhao et al., IEEE Trans Med Imaging 2019; 38:844-861), and has been demonstrated to be effective in enabling competitive estimation performance in numerical, phantom, and in vivo studies.

Note that the observed structure pertains to the formulations in (Zhao et al., IEEE Trans Med Imaging 2019; 38:844-861) and this disclosure-specifically, their design metrics and design constraints. It is worth emphasizing that if the design metric and/or constraints are changed, there is no guarantee that the same structure will appear. For example, the flip angle constraint could be replaced by a direct SAR constraint, which is linearly proportional to the square of the FAs and inversely proportional to the TRs (Prost et al., SAR reduced pulse sequences. Magn Reson Imaging 1988; 6:125-130; Brown et al., Magnetic Resonance Imaging: Physical Principles and Sequence Design. John Wiley & Sons, 2014). In this case, the resulting formulation would include a highly nonlinear constraint, and it would be interesting to examine the structure of the solution and the further utility of using B-spline subspace constraints for improved computational efficiency.

Additionally, it is important to gain an in-depth understanding of the structure arising in solving the OED problem from different perspectives using different approaches. While some spin physics perspectives (e.g., spin-coherence arguments in (Assländer et al., Cloos M A, Optimized quantification of spin relaxation times in the hybrid state. Magn Reson Med 2019; 82:1385-1397; Cloos et al., Multiparametric imaging with heterogeneous radiofrequency fields. Nat Commun 2016; 7:12445-12445)) are intuitive and may help understand the structure, the problem can be approached from an optimal control viewpoint. Specifically, the OED problem for MR Fingerprinting can be formulated as an optimal control problem with which the binary structure of the TR sequence could be characterized using bang-bang control (Lewis et al., Optimal Control. New York, NY: John Wiley & Sons, 2012). However, understanding the structure of the FA sequence remains an open problem that requires further theoretical study.

The present experimental example shows that the class of B-spline basis functions has good representation power and flexibility to represent the desired structure in the OED. In particular, by controlling the underlying knot sequences and the degree of the spline functions, the B-spline representations represent different structures associated with the flip angle and repetition time sequences. In general, the function classes chosen to represent the data acquisition parameter sequences depend on the structure enforced in the OED problem. Besides B-spline basis functions, it is understood that there are alternative function classes (e.g., wavelets, radial basis, Fourier basis) that may in some embodiments be well suited for the OED problem.

Note that the performance of the disclosed approach depends on the spline subspaces, which are governed by the knot sequences and degrees of the B-spline basis functions. In general, the selection of the B-spline parameters balances the dimensionality and representation power of the subspaces, and also allows for incorporating prior knowledge on the desired structure of the acquisition sequences. In the disclosed methods and systems, these parameters were manually selected to ensure a good trade-off between computational efficiency and estimation accuracy, while incorporating the observed optimal structure (e.g., the binary structure of the TR sequence (Zhao et al., IEEE Trans Med Imaging 2019; 38:844-861)) into the experimental design. More details regarding the impact of B-spline parameters on the performance of the disclosed approach are included in the second Experimental Example below.

The disclosed approach was initialized using the acquisition parameters from conventional MR Fingerprinting (Jiang et al., Magn Reson Med 2015; 74:1621-1631), which resulted in good performance as illustrated in the Results section. Note that the disclosed approach involves solving a nonconvex optimization problem, and its performance generally depends on the initialization of the algorithm. In Experimental Example #3below, the performance of the disclosed approach was evaluated with different initialization schemes. It was observed that the initialization of the flip angle sequence plays a dominant role in shaping the optimized acquisition parameters, and that among the schemes evaluated, the initialization from conventional MR Fingerprinting leads to the best performance. In some embodiments, for solving a nonconvex optimization problem in the disclosed approach, a multi-start strategy is used.

The SQP algorithm was applied to solve an OED problem whose computational bottleneck, i.e., the gradient evaluation with finite differences, significantly benefits from the reduced search space enabled by the disclosed subspace constraints. It is worth noting that the benefits of the disclosed constraints go beyond this specific gradient evaluation method. In one embodiment, the improvement of the computational efficiency for the SQP algorithm is demonstrated with another widely used gradient calculation method, i.e., automatic differentiation (Lee et al., Buonincontri G, Hargreaves B A, Flexible and efficient optimization of quantitative sequences using automatic differentiation of Bloch simulations. Magn Reson Med 2019; 82:1438-1451; Nocedal & Wright, Numerical Optimization. New York: Springer, 2nd edition, 2006).

As an alternative approach to numerical differentiation, automatic differentiation can be used in some embodiments to calculate the gradient of an objective function in optimization (J. Nocedal and S. J. Wright, Numerical Optimization, 2nd ed. New York: Springer, 2006; A. G. Baydin, et al., “Automatic differentiation in machine learning: a survey,” J. Mach. Learn. Res., vol. 18, 2018.) and was previously employed for the optimization of acquisition parameter sequences in MR Fingerprinting (P. K. Lee, et al., “Flexible and efficient optimization of quantitative sequences using automatic differentiation of Bloch simulations,” Magn. Reson. Med., vol. 82, pp. 1438-1451, 2019.) Here it was demonstrated that the algorithm, i.e., sequential quadratic programming, with automatic differentiation also benefits from the proposed subspace constraints.

Specifically, the OED problem in Equation 13 was solved using the sequential quadratic programming (SQP) algorithm for which the gradient was calculated using the reverse-mode automatic differentiation implementation from the Deep Learning Toolbox in MATLAB®. The algorithm was run on the computational platform described above, and, for simplicity, only a single CPU core of the server was used, as in P. K. Lee, et al., Magn. Reson. Med., vol. 82, pp. 1438-1451, 2019. Table 2 below shows the runtimes (in minutes) of solving the OED problems associated with Optimized-I, Optimized-II, and the disclosed approach with the sequential quadratic programming (SQP) algorithm with automatic differentiation for different acquisition lengths. As can be seen, the disclosed approach provides a significant improvement in computational efficiency over Optimized-I and Optimized-II for all the acquisition lengths. Finally, as expected, both automatic differentiation and numerical differentiation yield the same optimized acquisition parameter sequences.

TABLE 2

| Method | 300 | 400 | 500 | 600 |
|---|---|---|---|---|
| Optimized-I | 228.06 | 493.54 | 893.55 | 1125.68 |
| Optimized-II | 187.00 | 275.05 | 965.14 | 1094.39 |
| Disclosed | 21.60 | 31.96 | 38.36 | 59.38 |

Also note that the spline-based representations could be advantageous over other OED problems apart from the CRB-based experimental design. For example, Hu et al. presented an OED approach for MR Fingerprinting (Hu et al., Sequence design for fast and robust MR fingerprinting scans using quantum optimization. In: Proc. Int. Symp. Magn. Reson. Med., 2021. p. 169) that employed a spline-based representation for a supervised learning formulation with a stochastic optimization algorithm. Although a reduced search space generally brings computational benefits, the improvement is certainly problem and/or algorithm specific.

The present disclosure uses ML reconstruction to illustrate the estimation performance enabled by the CRB-based OED. However, the benefits of the CRB-based experimental design are not tied to any specific reconstruction algorithm because the CRB, as an estimation-theoretic bound, is independent of the reconstruction process. This is generally different from the emerging supervised learning based OED approaches (Heesterbeek et al., Sequence optimisation for multi-component analysis in magnetic resonance fingerprinting. In: Proc. Int. Symp. Magn. Reson. Med., 2021. p. 1561; Hu et al., Sequence design for fast and robust MR fingerprinting scans using quantum optimization. In: Proc. Int. Symp. Magn. Reson. Med., 2021. p. 169; Loktyushin et al., MRzero-Automated discovery of MRI sequences using supervised learning. Magn Reson Med 2021; 86:709-724), which often incorporate a specific reconstruction algorithm into an objective function that performs empirical risk minimization. In practice, it was observed that a variety of reconstruction algorithms, including both model-based (Zhao B, Model-based iterative reconstruction for magnetic resonance fingerprinting. In: Proc. Int. Conf. Image Process., 2015. pp. 3392-3396; Doneva et al., Matrix completion-based reconstruction for undersampled magnetic resonance fingerprinting data. Magn Reson Med 2017; 41:41-52; Zhao et al., Improved magnetic resonance fingerprinting reconstruction with low-rank and subspace modeling. Magn Reson Med 2018; 79:933-942; Mazor et al., Low-rank magnetic resonance fingerprinting. Med Phys 2018; 45:4066-4084) and data-driven methods (Cohen et al., MR fingerprinting Deep Reconstruction NEtwork (DRONE). Magn Reson Med 2018; 80:885-894; Fang et al., Deep learning for fast and spatially constrained tissue quantification from highly accelerated data in magnetic resonance fingerprinting. IEEE Trans Med Imaging 2019; 38:2364-2374), can benefit from the CRB-based experimental design, given the SNR benefit such design provides.

The CRB-based OED focuses on minimizing the variance of the MR tissue parameter estimates, although the overall estimation performance also depends on the bias of the tissue parameters. In numerical simulations, it was demonstrated that the disclosed approach yielded a similar level of bias as the conventional experiment and the state-of-the-art approaches. However, assessing the bias for experimental studies, and, in particular, in vivo studies, can be substantially more complex, since a variety of potential model mismatches (e.g., tissue microstructure, acquisition imperfections, etc.) in real imaging experiments could complicate the bias evaluation, and in many cases, finding a viable ground truth for the bias evaluation can be very challenging (Stikov et al., On the accuracy of T1 mapping: Searching for common ground. Magn Reson Med 2015; 73:514-522). A thorough investigation of the bias for experimental studies remains an open problem for MR Fingerprinting.

It is also worth pointing out several future extensions of the disclosed approach. First, this work has demonstrated the efficacy of the disclosed approach for optimizing the FAs and TRs for a representative MR Fingerprinting sequence, i.e., the IR-FISP sequence. The disclosed approach can also be applied or extended to include other acquisition parameters (e.g., TE) and design other MR Fingerprinting pulse sequences (Ma et al., Magnetic resonance fingerprinting. Nature 2013; 495:187-192; Cloos et al., Multiparametric imaging with heterogeneous radiofrequency fields. Nat Commun 2016; 7:12445-12445; Jiang et al., MR fingerprinting using the quick echo splitting NMR imaging technique. Magn Reson Med 2017; 77:979-988; Cui et al., A multi-inversion-recovery magnetic resonance fingerprinting for multi-compartment water mapping. Magn Reson Imaging 2021; 81:82-87). In particular, an interesting extension is to design MR Fingerprinting sequences with multiple inversion pulses (e.g., (Cui et al., A multi-inversion-recovery magnetic resonance fingerprinting for multi-compartment water mapping. Magn Reson Imaging 2021; 81:82-87)). In some such embodiments, the disclosed approach could be generalized from utilizing a single subspace to a union of subspaces. Here one additional subspace could be introduced to represent isolated inversion pulses, with the canonical basis as the basis functions. A sparsity constraint on the basis coefficients could further be imposed to control the number of inversion pulses. In some embodiments, this allows for the design of MR Fingerprinting sequences that have piecewise smooth FAs together with multiple isolated inversion pulses.

Second, although the effectiveness of employing a small number of representative tissues for the OED problem is demonstrated, the improved computational efficiency with the disclosed approach facilitates the inclusion of a more extensive range of tissue parameters into the OED problem or even a prior distribution of tissue parameters as in the Bayesian OED paradigm (Chaloner & Verdinelli, Bayesian experimental design: A review. Stat Sci 1995; 10:273-304). In some embodiments, the disclosed approach can also be extended to optimize MR Fingerprinting experiments with more complex tissue biophysical models, such as those used in myelin water imaging (Piredda et al., Probing myelin content of the human brain with MRI: A review. Magn Reson Med 2021; 85:627-652) and quantitative magnetization transfer imaging (Hilbert et al., Magnetization transfer in magnetic resonance fingerprinting. Magn Reson Med 2020; 84:128-141).

### Conclusions

The present experimental example and disclosure have introduced an efficient approach to solving the OED problem for MR Fingerprinting. Some embodiments of the disclosed approach employ B-spline based subspace constraints to incorporate the desired structure of data acquisition parameters and reduce the search space of the OED problem. Some embodiments of the disclosed approach enable a two-order-of-magnitude improvement of the computational efficiency over the state-of-the-art approaches, while providing a comparable SNR benefit.

### Example #2—Impact of B-Spline Parameters on the Performance of the Disclosed Approach

The proposed subspace constraints are governed by the degrees and knots sequences of the B-spline basis functions. In the first Experimental Example above the effectiveness of the disclosed approach using one set of B-spline parameters is shown. Here the impact of the B-spline parameters on the performance of the proposed approach is examined.

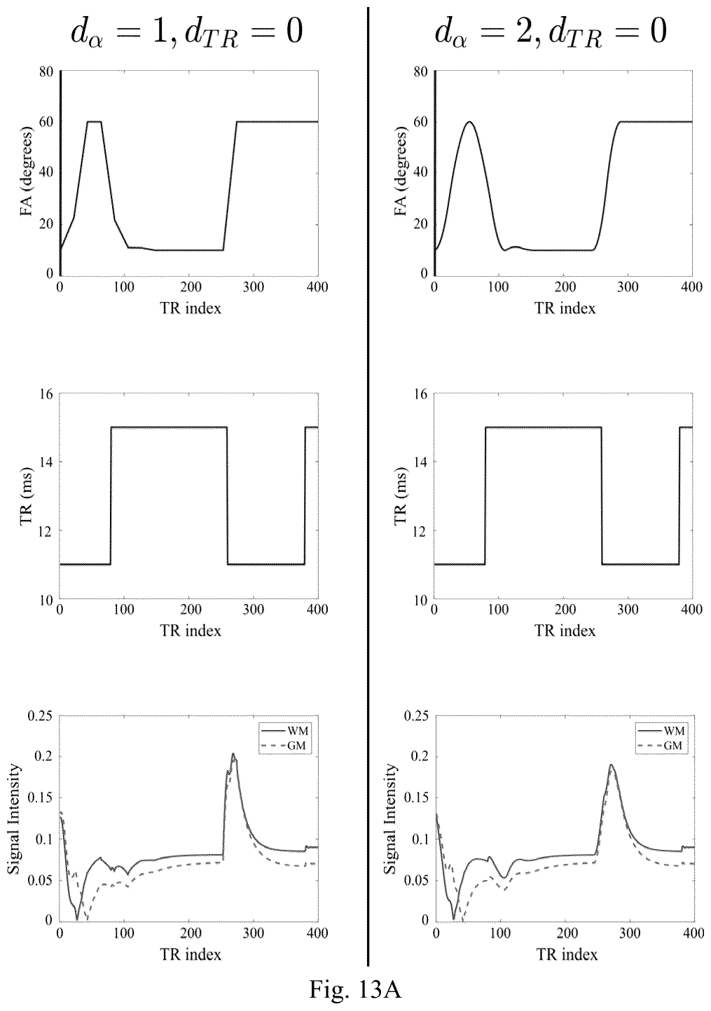

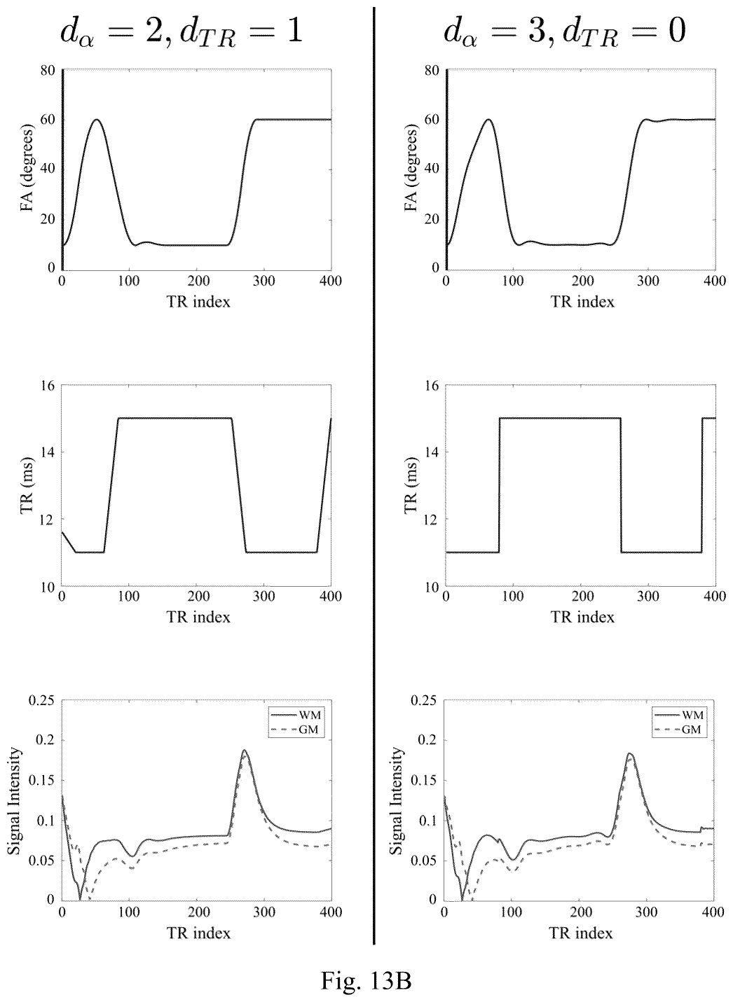

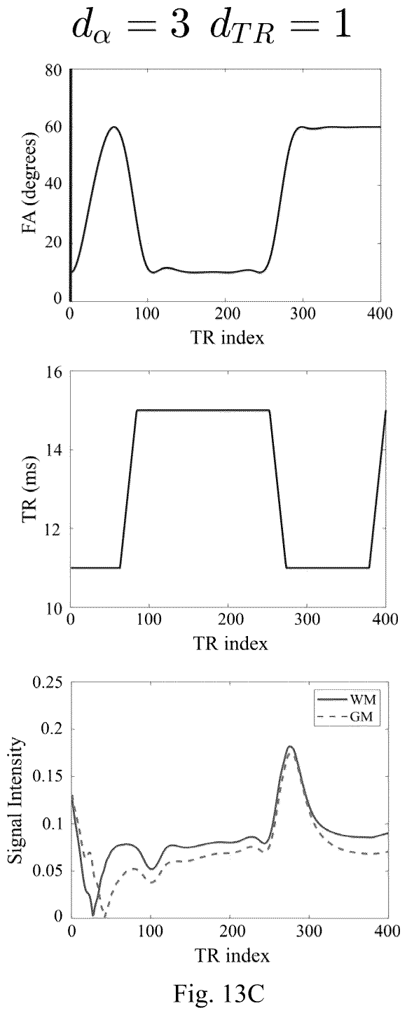

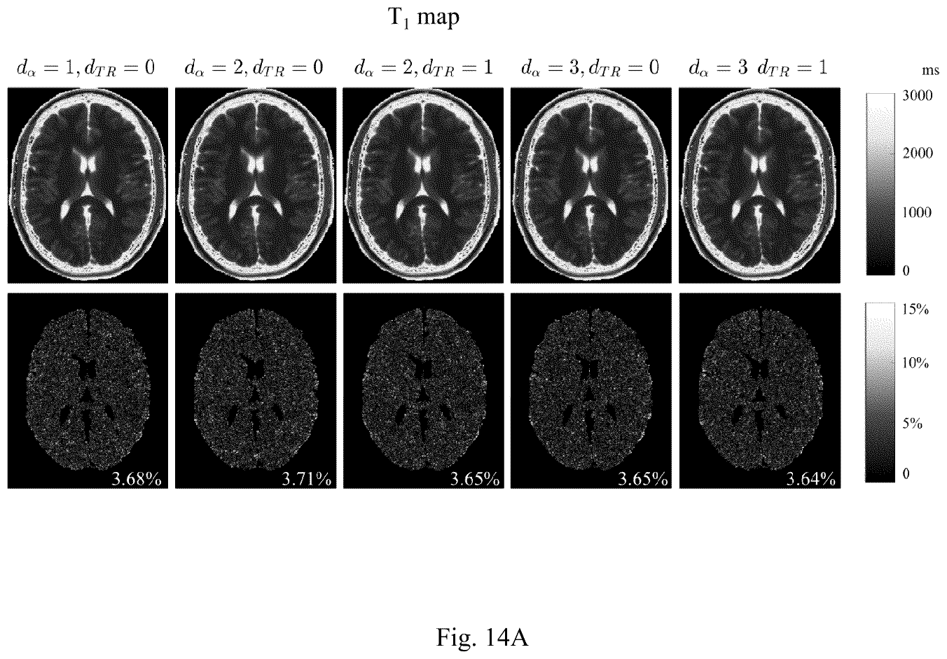

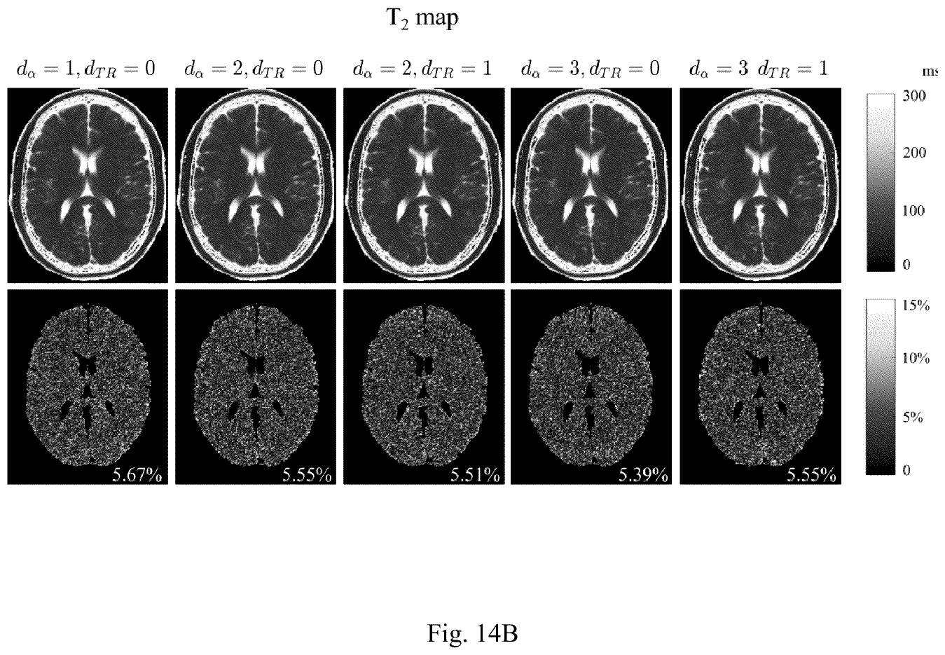

First, the impact of the choice of B-spline degree was evaluated. Specifically, different combinations of FA and TR B-spline degrees were used with the same knot sequence as the one used in the main text. FIG. 13A, FIG. 13B, and FIG. 13C show the resulting optimized FA and TR sequences and associated representative magnetization dynamics from the disclosed approach at N=400. Specifically, FIG. 13A shows FA at degrees of 1 and 2, and TR at degree 0. FIG. 13B shows FA at degree 2 and 3, and TR at degree 1 and 0, and FIG. 13C shows FA at degree 3 and TR at degree 1. As can be seen, the optimized acquisition parameter sequences and magnetization evolutions have similar structure for all choices of degrees considered. In FIG. 14A and FIG. 14B, the corresponding reconstructed T₁ and T₂ maps from the numerical simulations with N=400 and SNR=33 dB are shown. Here various combinations of B-spline degrees were used to represent the flip angle and repetition time sequences in the experimental design. As can be seen, in all cases, the performance is comparable to that of the disclosed approach with the choice of B-spline degree used in the first Experimental Example, which demonstrates the relative robustness with respect to this B-spline parameter.

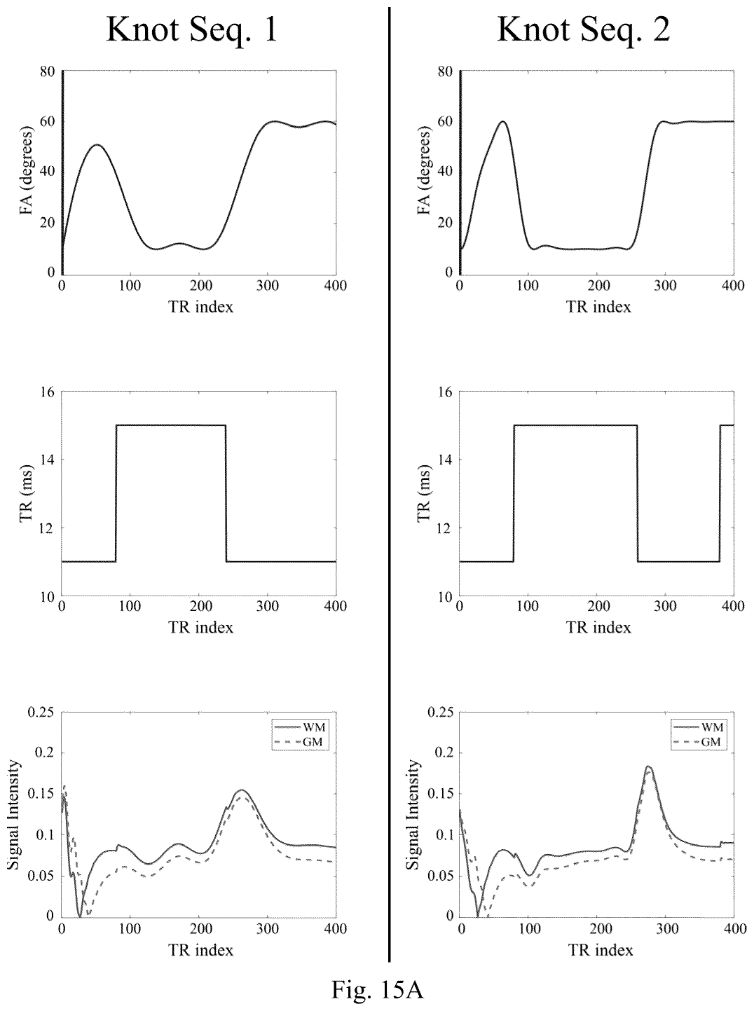

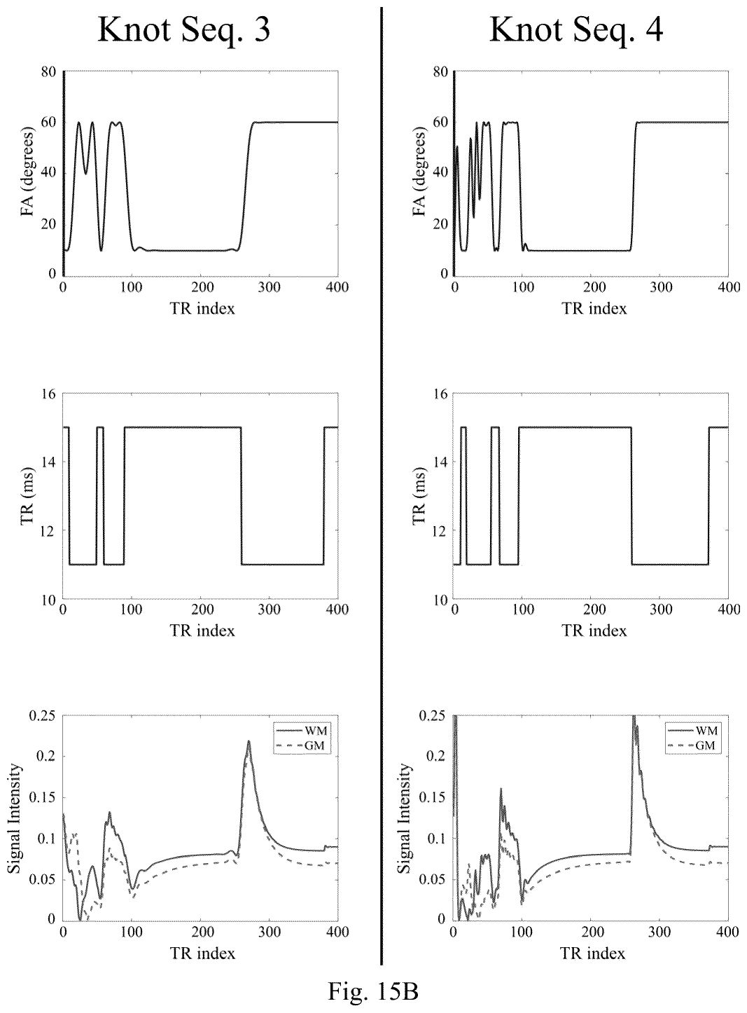

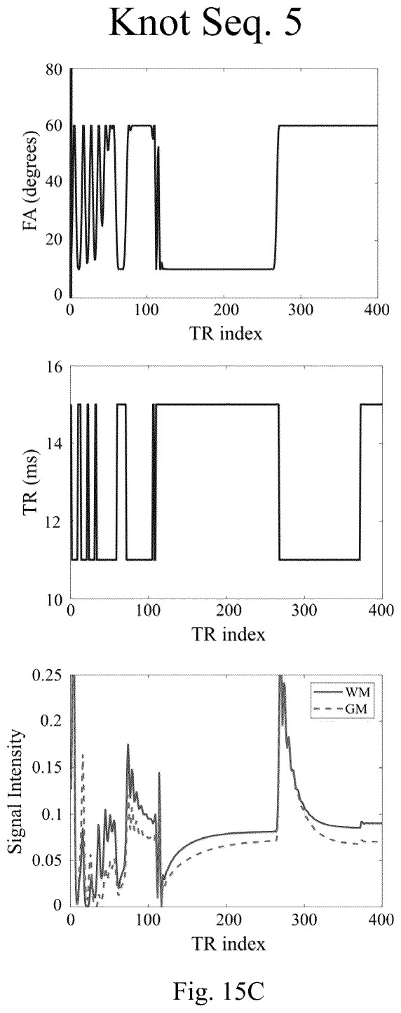

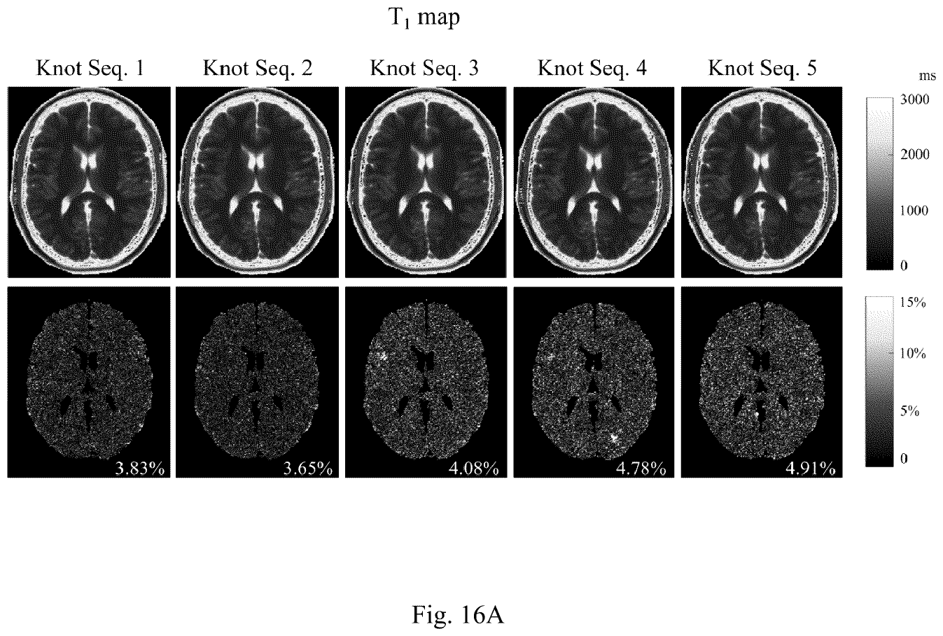

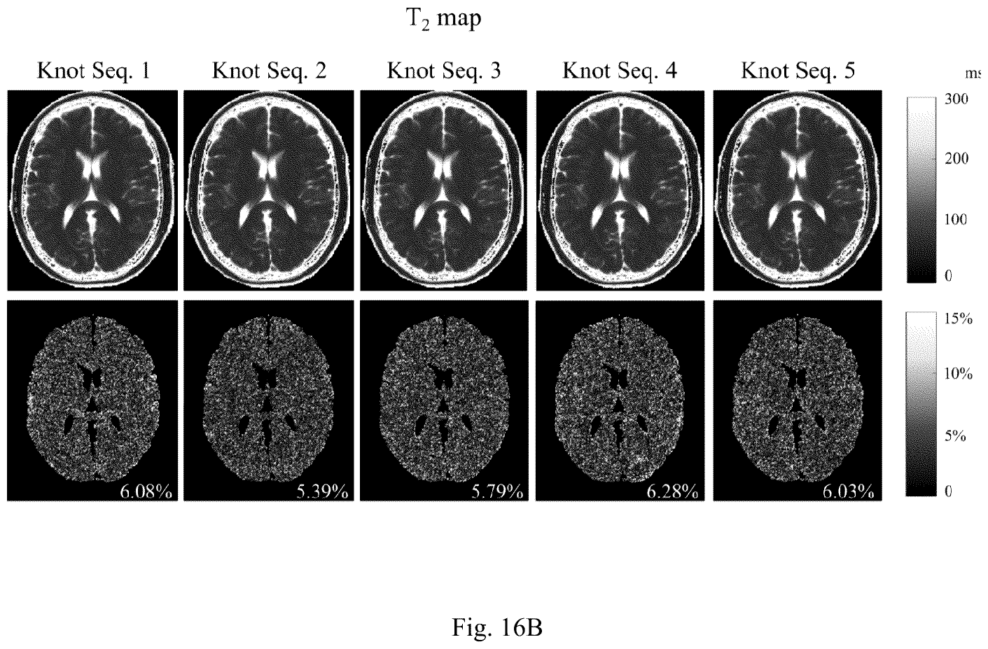

Second, the impact of the choice of B-spline knot sequence was evaluated. Here the same B-spline degrees (i.e., d_{α}=3 and d_{TR}=0) were used as in the first Experimental Example but different knot sequences were used. Specifically, five different uniformly spaced knot sequences were evaluated at acquisition length N=400 with corresponding B-spline subspaces of dimensions L_{α}=L_{TR}=10,20,40,100, and 200. FIG. 15A (10 and 20), FIG. 15B (40 and 100), and FIG. 15C (200) show the resulting acquisition parameters and associated magnetization dynamics. As can be seen, the choice of knot sequence plays a significant role in shaping the structure of the acquisition parameter sequences and magnetization evolutions. With a proper selection of the knot sequence, the desired magnetization evolutions can be obtained that lead to better estimation performance. The corresponding reconstructed T₁ and T₂ maps are shown in FIG. 16A and FIG. 16B. Specifically, Knot Seq. 1, 2, 3, 4, and 5 are uniformly spaced knot vectors with associated B-spline subspaces of dimension L_{α}=L_{TR}=10,20,40,100, and 200, respectively. As can be seen, the knot sequence plays an important role in determining the structure of the B-spline subspaces and the resulting estimation performance.

Furthermore, as illustrated in FIG. 15A, FIG. 15B, and FIG. 15C, as the dimension of the B-spline subspaces approaches the acquisition length, the optimized acquisition parameters approach those from Optimized-I. When the dimension of the B-spline subspace is equal to the acquisition length, the solution space of the proposed approach is equal to that of Optimized-I, and with equivalent initializations, it can be shown that the solution of the proposed approach is the same as the one for Optimized-I.

### Example #3—Impact of Initialization on the Experimental Design

Next, the impact of the initialization of the algorithm on the performance of the disclosed approach was evaluated. Specifically, the SQP algorithm was initialized by performing a least-squares fit of the B-spline coefficients to the following four acquisition parameter sequences:
(1) FA sequence and TR sequence both from conventional MR Fingerprinting (D. Ma, et al., “Magnetic resonance fingerprinting,” Nature, vol. 495, pp. 187-192, 2013.) (i.e., the one used in the first Experimental Example)
(2) FA sequence from conventional MR Fingerprinting and TR sequence with each TRs from the uniform distribution on [11 ms, 15 ms]
(3) FA sequence with FAs from the uniform distribution on the interval [10°, 60°] and TR sequence from conventional MR Fingerprinting
(4) FA sequence with FAs from the uniform distribution on the interval [10°, 60°] and TR sequence with TRs from the uniform distribution on [11 ms, 15 ms].

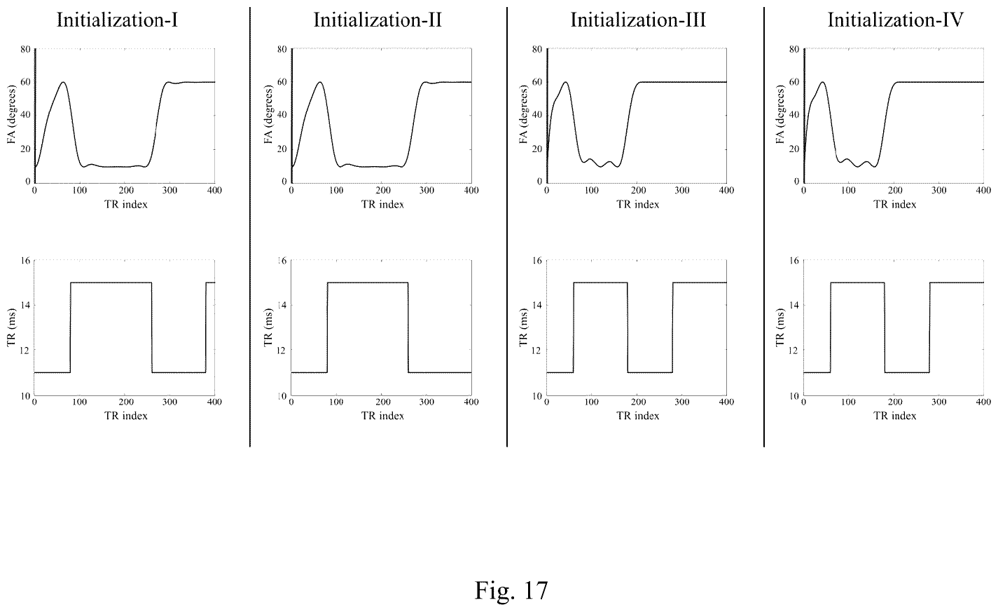

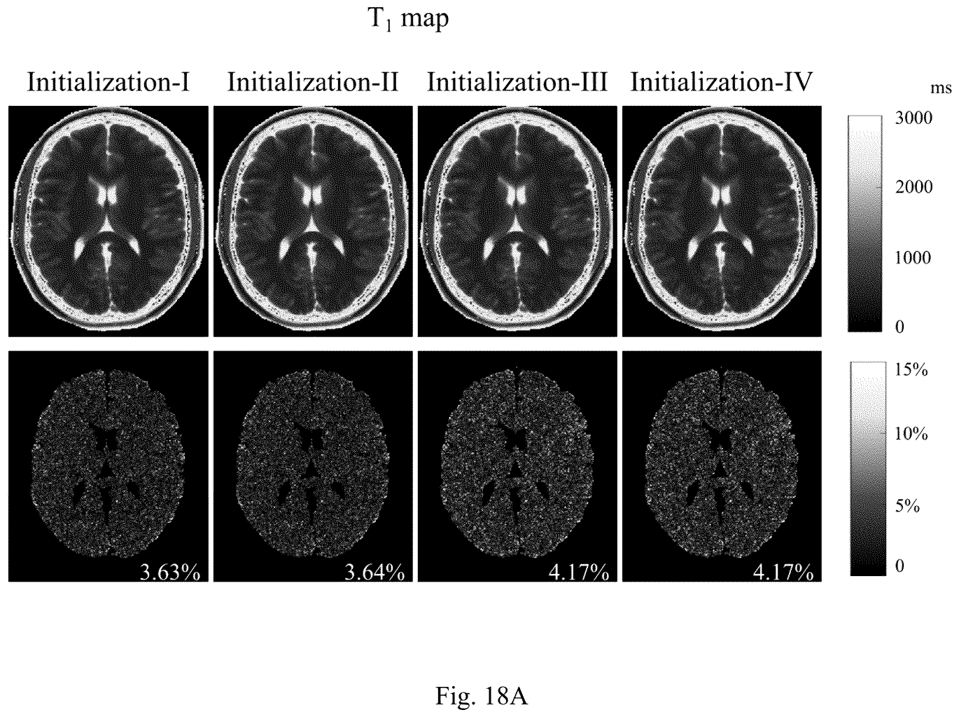

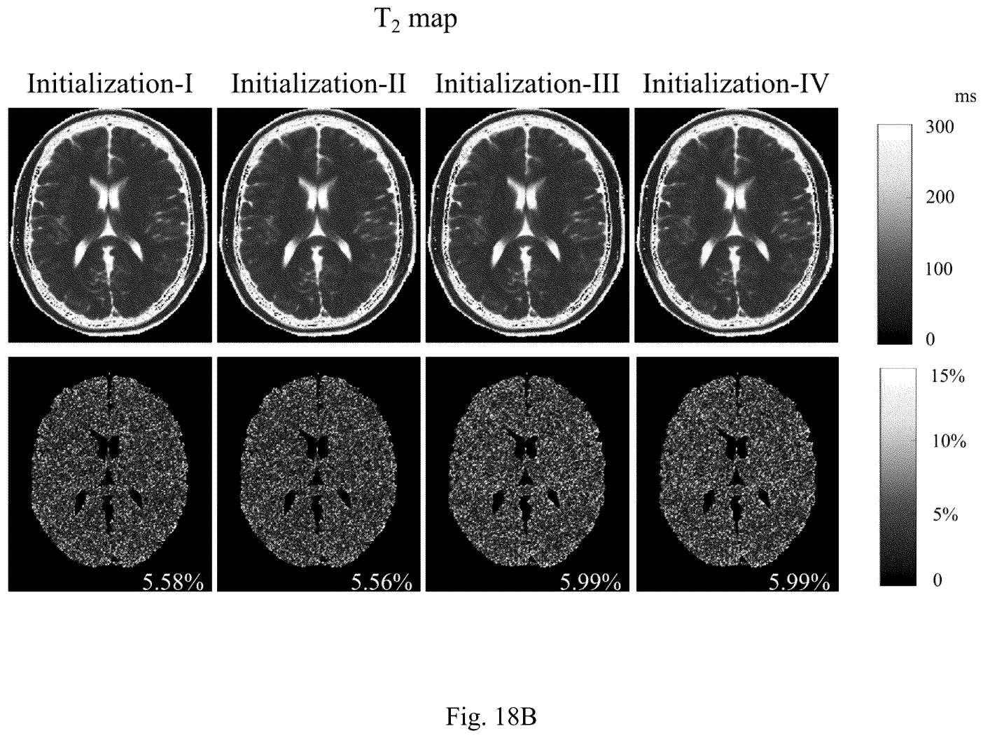

FIG. 17 shows the optimized FA and TR sequences using the above initializations as well as the cost function values associated with these solutions. As can be seen, the solution of the algorithm depends on the initialization. In particular, it was observed that the initialization of the FA sequence plays a dominant role in shaping the optimized acquisition parameters. Note that the cost function values for the solution using the four initializations are 3.75, 3.75, 3.88, and 3.88, respectively Compared with other initializations, the two initialization schemes using the FA sequence from the conventional MR Fingerprinting (Initialization-I and Initialization-II) result in the best cost function value. FIG. 18A and FIG. 18B show the reconstructed T₁ and T₂ maps using the acquisition sequences in FIG. 17 at acquisition length N=300 and SNR=33 dB, which illustrates the impact of these optimized acquisition parameters on the reconstructed tissue parameter maps.

### Example #4—Impact of the Acceleration Factor

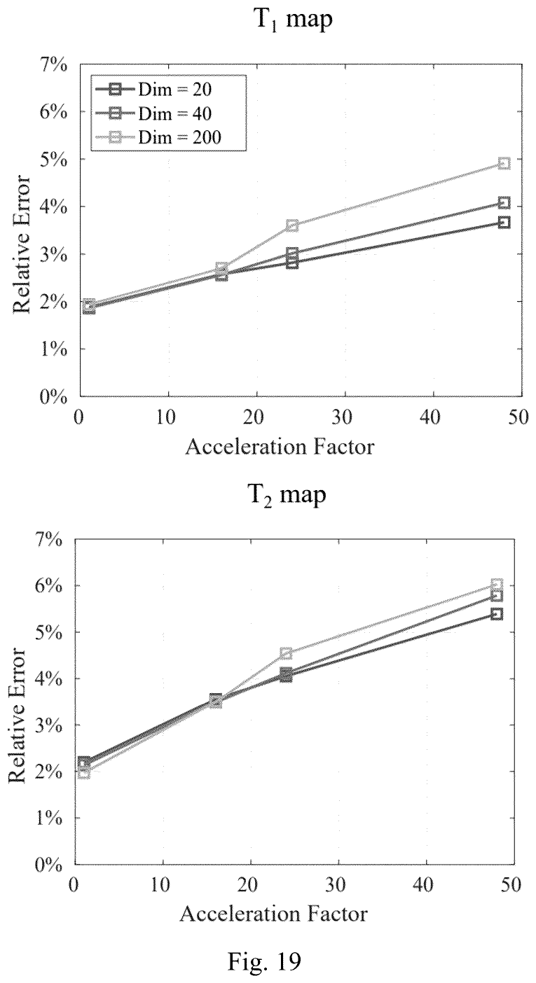

The performance of the OED was evaluated using various acceleration factors for different choices of spline subspace dimension. Specifically, spline subspaces of dimensions L_{α}=L_{TR}=20,40, and 200 were considered for MR Fingerprinting experiments with acceleration factors (AF)=1,16,24, and 48. Note that the case with L_{α}=L_{TR}=20 and AF=48 (i.e., one spiral interleave per TR) corresponds to the one shown in the first Experimental Example. FIG. 19 shows the resulting overall relative reconstruction errors for T₁ and T₂. using the OED with B-spline subspaces of dimension L_{α}=L_{TR}=20,40, and 200 and acceleration factors AF=1, 16, 24, and 48 at N=400 and SNR=33 dB. As can be seen, in a highly-undersampled regime (e.g., AF=48 or 24), enforcing the smoothness constraint in the OED is beneficial for improving the reconstruction performance. However, such benefit reduces as the acceleration factor decreases. Note that this is consistent with the data model used in the OED, which corresponds to a fully-sampled imaging experiment. Nonetheless, for an MR Fingerprinting experiment with a lower acceleration factor (e.g., AF=16) or even the fully-sampled case (i.e., AF=1), imposing the smoothness constraint in the OED does not appear to compromise the reconstruction performance much, which is highly desirable in practice. Note that the observations for the fully-sampled spiral MRF experiments are consistent with early observations for the fully-sampled Cartesian imaging experiment (see B. Zhao, et al., IEEE Trans. Med Imaging, vol. 38, no. 3, pp. 844-861, 2019.)

Despite this insightful illustration in numerical simulations, it is worth noting that MR Fingerprinting is a transient-state imaging technique in which there is no steady-state for magnetizations. As a result, having a lower acceleration factor than 48 (equivalently, acquiring multiple spiral interleaves per TR) requires a substantial wait time (e.g., about 10 seconds) between different repetitions of acquisitions to ensure that the magnetization returns to the thermal equilibrium (a more detailed description of fully-sampled MR Fingerprinting experiments can be found in the early work B. Zhao, et al., IEEE Trans. Med Imaging, vol. 38, no. 3, pp. 844-861, 2019). In practice, this waiting time can be even longer than the actual acquisition time (e.g., about 5 seconds for N=400), which makes MR Fingerprinting experiments with a low AF less practically appealing. Thus, only the highly-undersampled MR Fingerprinting experiment with AF=48 was considered in the first Experimental Example.

### Example #5—Different Solution Algorithms

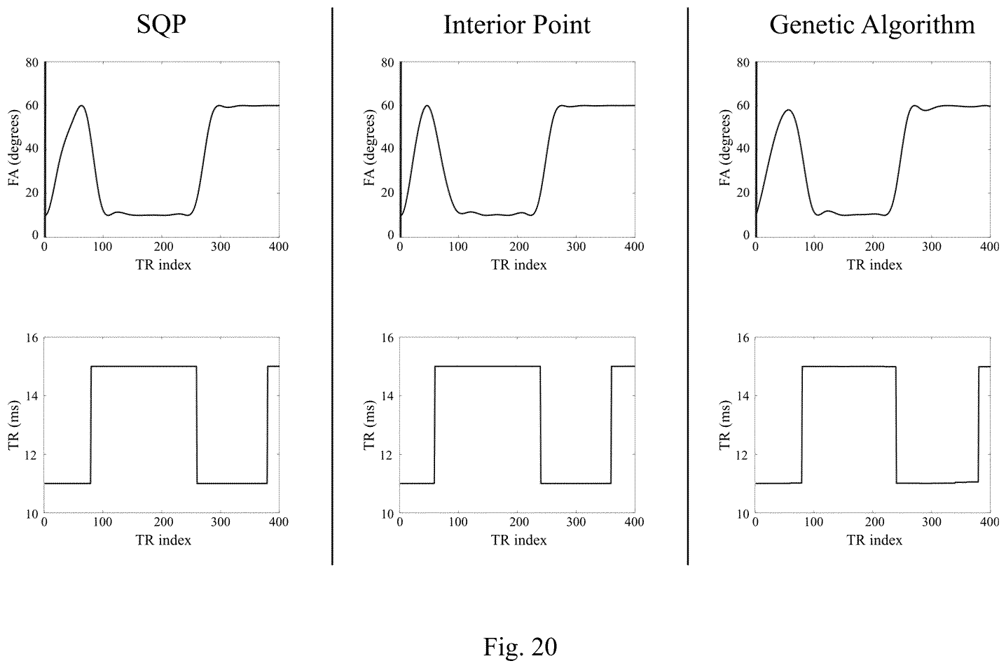

In the first Experimental Example, the SQP algorithm was used to solve the proposed formulation in Equation 13, which is a constrained nonlinear optimization problem with both bound constraints and linear inequality constraints. Here the results of solving this optimization problem using two alternative solution algorithms are shown. The first alternative algorithm was another deterministic nonlinear programming algorithm, i.e., an interior-point algorithm (J. Nocedal and S. J. Wright, Numerical Optimization, 2nd ed. New York: Springer, 2006; A. Wächter and L. Biegler, “On the implementation of an interior-point filter line-search algorithm for large-scale nonlinear programming,” Math. Program., vol. 106, no. 1, pp. 25-57, 2006.) which has the same local convergence guarantee as the disclosed approach. Second, a stochastic nonlinear programming algorithm, i.e., a genetic algorithm, was applied which has completely different convergence behavior. More specifically, the same initialization was used for the interior-point algorithm as the disclosed approach, which is used to confirm the convergence of the disclosed approach. For the genetic algorithm, a uniform random initialization was used. FIG. 20 shows the optimized acquisition parameters from the SQP algorithm, the interior-point algorithm, and the genetic algorithm. The cost function values associated with the three solutions are 3.75, 3.77, and 3.77, respectively. As can be seen, three dramatically different algorithms produce highly similar FA and TR sequences, which is remarkable. Although the cost function values associated with the optimal solutions from the three algorithms are very similar, the SQP algorithm used in the present disclosure produces the best cost function value.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### REFERENCES

The following publications are incorporated herein by reference in their entirety:
Ma D, Gulani V, Seiberlich N, Liu K, Sunshine J L, Duerk J L, Griswold M A, Magnetic resonance fingerprinting. Nature 2013; 495:187-192.
Chen Y, Jiang Y, Pahwa S, Ma D, Lu L, Twieg M D, Wright K L, Seiberlich N, Griswold M A, Gulani V, M R fingerprinting for rapid quantitative abdominal imaging. Radiology 2016; 279:278-286.
Badve C, Yu A, Dastmalchian S, Rogers M, Ma D, Jiang Y, Margevicius S, Pahwa S, Lu Z, Schluchter M, Sunshine J, Griswold M A, Sloan A, Gulani V, M R fingerprinting of adult brain tumors: Initial experience. Am J Neuroradiol 2017; 38:492-499.
Yu A, Badve C, Ponsky L E, Pahwa S, Dastmalchian S, Rogers M, Jiang Y, Margevicius S, Schluchter M, Tabayoyong W, Abouassaly R, McGivney D, Griswold M A, Gulani V, Development of a combined M R fingerprinting and diffusion examination for prostate cancer. Radiology 2017; 283:729-738.
Liu Y, Hamilton J, Rajagopalan S, Seiberlich N, Cardiac magnetic resonance fingerprinting: Technical overview and initial results. JACC: Cardiovasc Imaging 2018; 11:1837-1853.
Chen Y, Panda A, Pahwa S, Hamilton J I, Dastmalchian S, McGivney D F, Ma D, Batesole J, Seiberlich N, Griswold M A, Plecha D, Gulani V, Three-dimensional M R fingerprinting for quantitative breast imaging. Radiology 2019; 290:33-40.
Cloos M A, Assländer J, Abbas B, Fishbaugh J, Babb J S, Gerig G, Lattanzi R, Rapid radial T1 and T2 mapping of the hip articular cartilage with magnetic resonance fingerprinting. J Magn Reson Imaging 2019; 50:810-815.
Kiselev V G, Körzdörfer G, Gall P, Toward quantification: Microstructure and magnetic resonance fingerprinting. Investig Radiol 2021; 56:1-9.
Jaubert O, Cruz G, Bustin A, Hajhosseiny R, Nazir S, Schneider T, Koken P, Doneva M, Rueckert D, Masci P G, Botnar R M, Prieto C, T1, T2, and fat fraction cardiac M R fingerprinting: Preliminary clinical evaluation. J Magn Reson Imaging 2021; 53:1253-1265.
Zhao B, Haldar J P, Setsompop K, Wald L L, Optimal experiment design for magnetic resonance fingerprinting. In: Proc. IEEE Eng. Med. Bio. Conf., 2016. pp. 453-456.
Zhao B, Haldar J P, Setsompop K, Wald L L, Towards optimized experiment design for magnetic resonance fingerprinting. In: Proc. Int. Symp. Magn. Reson. Med., 2016. p. 2835.
Zhao B, Haldar J P, Liao C, Ma D, Jiang Y, Griswold M A, Setsompop K, Wald L L, Optimal experiment design for magnetic resonance fingerprinting: Cramér-Rao bound meets spin dynamics. IEEE Trans Med Imaging 2019; 38:844-861.
Jones J, Hodgkinson P, Barker A, Hore P, Optimal sampling strategies for the measurement of spin-spin relaxation times. J Magn Reson 1996; 113:25-34.
Deoni S C L, Peters T M, Rutt B K, Determination of optimal angles for variable nutation proton magnetic spin-lattice, T1, and spin-spin, T2, relaxation times measurement. Magn Reson Med 2004; 51:194-199.
Fleysher L, Fleysher R, Liu S, Zaaraoui W, Gonen O, Optimizing the precision-per-unit-time of quantitative M R metrics: examples for T1, T2, and DTI. Magn Reson Med 2007; 57:380-387.
Akçakaya M, Weingärtner S, Roujol S, Nezafat R, On the selection of sampling points for myocardial T1 mapping. Magn Reson Med 2015; 73:1741-1753.
Whitaker S T, Nataraj G, Nielsen J, Fessler J A, Myelin water fraction estimation using small-tip fast recovery MRI. Magn Reson Med 2020; 84:1977-1990.
Maidens J, Packard A, Arcak M, Parallel dynamic programming for optimal experiment design in nonlinear systems. In: Proc. IEEE Conf. Decis. Control, 2016. pp. 2894-2899.
Lee P K, Watkins L E, Anderson T I, Buonincontri G, Hargreaves B A, Flexible and efficient optimization of quantitative sequences using automatic differentiation of Bloch simulations. Magn Reson Med 2019; 82:1438-1451.
Assländer J, Lattanzi R, Sodickson D K, Cloos M A, Optimized quantification of spin relaxation times in the hybrid state. Magn Reson Med 2019; 82:1385-1397.
Lahiri A, Fessler J A, Hernandez-Garcia L, Optimizing MRF-ASL scan design for precise quantification of brain hemodynamics using neural network regression. Magn Reson Med 2020; 83:1979-1991.
Heesterbeek D, Vos F, Gijzen M, Nagtegaal M, Sequence optimisation for multi-component analysis in magnetic resonance fingerprinting. In: Proc. Int. Symp. Magn. Reson. Med., 2021. p. 1561.
Scope Crafts E, Zhao B, An efficient approach to optimal design of M R fingerprinting experiments with B-splines. In: Proc. Int. Symp. Magn. Reson. Med., 2021. p. 1566.
Jiang Y, Ma D, Seiberlich N, Gulani V, Griswold M A, M R fingerprinting using fast imaging with steady state precession (FISP) with spiral readout. Magn Reson Med 2015; 74:1621-1631.
Höllig K, Hörner J, Approximation and Modeling with B-Splines. Philadelphia, P A: Society for Industrial and Applied Mathematics, 2013.
Schumaker L, Spline Functions: Basic Theory. Cambridge: Cambridge University Press, 2007.
Heath M T, Scientific Computing: An Introductory Survey. Philadelphia, P A: Society for Industrial and Applied Mathematics, 2018.
Hastie T, Tibshirani R, Friedman J, The Elements of Statistical Learning: Data Mining, Inference, and Prediction. New York: Springer, 2009.
Fey M, Lenssen J E, Weichert F, Müller H, SplineCNN: Fast geometric deep learning with continuous B-spline kernels. In: Proc. IEEE/CVF Conf. Comput. Vis. Pattern Recognit., 2018. pp. 869-877.
Unser M, Aldroub A, Eden M, B-spline signal processing: Part I-theory. IEEE Trans Signal Process 1993; 41:821-833.
Unser M, Aldroub A, Eden M, B-spline signal processing: Part I I-efficient design and applications. IEEE Trans Signal Process 1993; 41:834-848.
Unser M, Splines: A perfect fit for signal and image processing. IEEE Signal Process Mag 1999; 16:22-38.
Rueckert D, Sonoda L, Hayes C, Hill D, Leach M, Hawkes D, Nonrigid registration using free-form deformations: Application to breast M R images. IEEE Trans Med Imaging 1999; 18:712-721.
Hao S, Fessler J A, Noll D C, Nielsen J F, Joint design of excitation k-space trajectory and R F pulse for small-tip 3D tailored excitation in MRI. IEEE Trans Med Imaging 2016; 35:468-479.
de Boor C, On calculating with B-splines. J Approx Theory 1972; 6:50-62.
Nocedal J, Wright S J, Numerical Optimization. New York: Springer, 2nd edition, 2006.
Spall J C, Introduction to Stochastic Search and Optimization: Estimation, Simulation, and Control. New York, N Y: John Wiley & Sons, 2003.
Shkarin P, Spencer R G, Time domain simulation of Fourier imaging by summation of isochromats. Int J Imaging Syst Technol 1997; 8:419-426.
Malik S J, Sbrizzi A, Hoogduin H, Hajnal J V, Equivalence of EPG and isochromat-based simulation of M R signals. In: Proc. Int. Symp. Magn. Reson. Med., 2016. p. 3196.
Collins D L, Zijdenbos A P, Kollokian V, Sled J G, Kabani N J, Holmes C J, Evans A C, Design and construction of a realistic digital brain phantom. IEEE Trans Med Imaging 1998; 17:463-468.
Fessler J A, Sutton B P, Nonuniform fast Fourier transforms using min-max interpolation. IEEE Trans Signal Process 2003; 51:560-574.
Zhao B, Setsompop K, Ye H, Cauley S F, Wald L L, Maximum likelihood reconstruction for magnetic resonance fingerprinting. IEEE Trans Med Imaging 2016; 35:1812-1823.
Ma D, Coppo S, Chen Y, McGivney D F, Jiang Y, Pahwa S, Gulani V, Griswold M A, Slice profile and B_1 corrections in 2D magnetic resonance fingerprinting. Magn Reson Med 2017; 78:1781-1789.
Nataraj G, Nielsen J F, Fessler J A, Optimizing M R scan design for model-based T1, T2 estimation from steady-state sequences. IEEE Trans Med Imaging 2017; 36:467-477.
Prost J E H, Wehrli F W, Drayer B, Froelich J, Hearshen D, Plewes D, SAR reduced pulse sequences. Magn Reson Imaging 1988; 6:125-130.
Brown R W, Cheng Y C N, Haacke E M, Thompson M R, Venkatesan R, Magnetic Resonance Imaging: Physical Principles and Sequence Design. John Wiley & Sons, 2014.
Cloos M A, Knoll F, Zhao T, Block K T, Bruno M, Wiggins G C, Sodickson D K, Multiparametric imaging with heterogeneous radiofrequency fields. Nat Commun 2016; 7:12445-12445.
Lewis F L, Vrabie D, Syrmos V, Optimal Control. New York, N Y: John Wiley & Sons, 2012.
Hu S, Rozada I, Boyacioglu R, Jordan S, Huang S, Troyer M, Griswold M A, McGivney D, Ma D, Sequence design for fast and robust M R fingerprinting scans using quantum optimization. In: Proc. Int. Symp. Magn. Reson. Med., 2021. p. 169.
Loktyushin A, Herz K, Dang N, Glang F, Deshmane A, Weinmüller S, Doerfler A, Schölkopf B, Scheffler K, Zaiss M, MRzero-Automated discovery of MRI sequences using supervised learning. Magn Reson Med 2021; 86:709-724.
Zhao B, Model-based iterative reconstruction for magnetic resonance fingerprinting. In: Proc. Int. Conf. Image Process., 2015. pp. 3392-3396.
Doneva M, Amthor T, Koken P, Sommer K, Börnert P, Matrix completion-based reconstruction for undersampled magnetic resonance fingerprinting data. Magn Reson Med 2017; 41:41-52.
Zhao B, Setsompop K, Adalsteinsson E, Gagoski B, Ye H, Ma D, Jiang Y, Grant P E, Griswold M A, Wald L L, Improved magnetic resonance fingerprinting reconstruction with low-rank and subspace modeling. Magn Reson Med 2018; 79:933-942.
Mazor G, Weizman L, Tal A, Eldar Y C, Low-rank magnetic resonance fingerprinting. Med Phys 2018; 45:4066-4084.
Cohen O, Zhu B, Rosen M S, M R fingerprinting Deep RecOnstruction NEtwork (DRONE). Magn Reson Med 2018; 80:885-894.
Fang Z, Chen Y, Liu M, Xiang L, Zhang Q, Wang Q, Lin W, Shen D, Deep learning for fast and spatially constrained tissue quantification from highly accelerated data in magnetic resonance fingerprinting. IEEE Trans Med Imaging 2019; 38:2364-2374.
Stikov N, Boudreau M, Levesque I R, Tardif C L, Barral J K, Pike G B, On the accuracy of T1 mapping: Searching for common ground. Magn Reson Med 2015; 73:514-522.
Jiang Y, Ma D, Jerecic R, Duerk J, Seiberlich N, Gulani V, Griswold M A, M R fingerprinting using the quick echo splitting NMR imaging technique. Magn Reson Med 2017; 77:979-988.
Cui D, Hui E S, Cao P, A multi-inversion-recovery magnetic resonance fingerprinting for multi-compartment water mapping. Magn Reson Imaging 2021; 81:82-87.
Chaloner K, Verdinelli I, Bayesian experimental design: A review. Stat Sci 1995; 10:273-304.
Piredda G F, Hilbert T, Thiran J P, Kober T, Probing myelin content of the human brain with MRI: A review. Magn Reson Med 2021; 85:627-652.
Hilbert T, Xia D, Block K T, Yu Z, Lattanzi R, Sodickson D K, Kober T, Cloos M A, Magnetization transfer in magnetic resonance fingerprinting. Magn Reson Med 2020; 84:128-141.

What is claimed is:

1. A method of performing a diagnostic scan of a subject, comprising:
defining a set of data acquisition parameter sequences;
defining a set of upper bounds and a set of lower bounds for each of the set of data acquisition parameter sequences;
defining a set of representative tissue parameters for a tissue of the subject;
selecting a set of basis functions;
calculating values for a set of basis function coefficients to yield a piecewise polynomial representation of each of the set of data acquisition parameter sequences within the sets of upper and lower bounds based on the set of desired tissue parameters; and
performing a diagnostic scan of the subject using the calculated data acquisition parameter sequences.

2. The method of claim 1, wherein the diagnostic scan is a magnetic resonance imaging (MRI) scan.

3. The method of claim 2, wherein the diagnostic scan is a magnetic resonance fingerprinting scan.

4. The method of claim 1, wherein the set of data acquisition parameter sequences are selected from flip angles, radiofrequency phases, repetition times, echo times, time-bandwidth products, or k-space sampling locations.

5. The method of claim 1, wherein the set of basis functions is selected from B-splines, wavelets, radial basis functions, or Fourier basis functions.

6. The method of claim 5, wherein the set of basis functions is a B-spline basis function.

7. The method of claim 6, further comprising the step of selecting a degree of the B-spline basis functions to use in the step of calculating the piecewise polynomial.

8. The method of claim 7, wherein the degree of the B-spline basis function is selected using a machine learning algorithm.

9. The method of claim 1, further comprising the steps of:
generating a set of possible values for each of the set of basis function coefficients;
iterating through each combination of the possible values in the set of possible values to generate a set of piecewise polynomials;
calculating a result of a simulated diagnostic scan for each piecewise polynomial in the set of piecewise polynomials to create a multidimensional set of results; and
calculating a maximum value among the multidimensional set of results using an algorithm to yield the piecewise polynomial representation;
wherein the step of calculating the values of the set of basis function coefficients to yield the piecewise polynomial comprises executing an iterative algorithm.

10. The method of claim 9, wherein the iterative algorithm is selected from a sequential quadratic programming algorithm, an interior point algorithm, a genetic algorithm, and a simulated annealing algorithm.

11. The method of claim 10, wherein the algorithm is a sequential quadratic programming algorithm.

12. The method of claim 9, wherein the algorithm comprises setting a tolerance threshold and terminating the algorithm when the norm of a gradient of the multidimensional set of results is less than the tolerance threshold.

13. The method of claim 9, wherein the simulated diagnostic scan is a Bloch simulation.

14. The method of claim 1, further comprising selecting a set of time offsets for the set of basis functions to calculate the piecewise polynomial.

15. The method of claim 1, wherein the tissue of the subject is selected from liver, spleen, kidney medulla, kidney cortex, kidney, skeletal muscle, fat, myocardium, abdomen, lungs, stomach, intestines, brain, or blood.

16. The method of claim 1, wherein the step of defining the set of upper and lower bounds comprises calculating at least one of the upper and lower bound based on a desired total acquisition time or a specific absorption rate.

17. The method of claim 1, wherein the step of calculating values for each of the set of basis function coefficients to yield a piecewise polynomial representation of each of the set of data acquisition parameters comprises constructing the piecewise polynomials using the Cox-de Boor recursion formula.
